(19) United States
(12) Patent Application Publication
(10) Pub. No.: US 2026/0132141 A1
(43) Pub. Date: May 14, 2026

(54) PROTEIN PHOSPHATASE 2A ACTIVATORS

(71) Applicants: University of Florida Research Foundation, Incorporated, Gainesville, FL (US); The Penn State Research Foundation, University Park, PA (US)

(72) Inventors: Arun K. Sharma, University Park, PA (US); Amandeep Singh, University Park, PA (US); Hannah Johnson, University Park, PA (US); Asif Raza, University Park, PA (US); Satya Narayan, Gainesville, FL (US)

(73) Assignees: University of Florida Research Foundation, Incorporated, Gainesville, FL (US); The Penn State Research Foundation, University Park, PA (US)

(21) Appl. No.: 19/118,534

(22) PCT Filed: Oct. 6, 2023
(86) PCT No.: PCT/US2023/076212

§ 371 Date: Apr. 4, 2025

Related U.S. Application Data
(60) Provisional application No. 63/413,753, filed on Oct. 6, 2022.

(51) Int. Cl. C07D 487/18; A61K 31/282; A61K 31/337; A61K 31/436; A61K 31/473; A61K 31/4745; A61K 31/475; A61K 31/496; A61K 31/513; A61K 31/517; A61K 31/519; A61K 31/58; A61K 31/704; A61K 31/7068; A61K 31/7072; A61K 47/54; A61P 35/00

(52) U.S. Cl. CPC C07D 487/18; A61K 31/282; A61K 31/337; A61K 31/436; A61K 31/473; A61K 31/4745; A61K 31/475; A61K 31/496; A61K 31/513; A61K 31/517; A61K 31/519; A61K 31/58; A61K 31/704; A61K 31/7068; A61K 31/7072; A61K 47/545; A61P 35/00

(57) ABSTRACT

Disclosed herein are compounds with protein phosphatase 2A agonizing activity. In some embodiments, the compounds may be used in the treatment of cellular disorders, including cancer. In some embodiments, the compounds are used to treat colorectal cancer, including FOLFOX resistant colorectal cancer.

### CROSS-REFERENCE TO RELATED APPLICATIONS

This application claims the benefit of and priority under 35 U.S.C. § 119(e) to U.S. Provisional Application No. 63/413,753, filed Oct. 6, 2022, titled PROTEIN PHOSPHATASE 2A ACTIVATORS, the contents of which are incorporated herewith by reference in their entirety.

### FIELD OF THE INVENTION

The invention relates to novel protein phosphate 2A (PP2A) activators.

### BRIEF DESCRIPTION OF THE DRAWINGS

FIG. 1 depicts the chemical structure of NSC49L and iHAP1.

FIG. 2 depicts the chemical synthesis of PP2A activators.

FIG. 3 depicts IC50 values of compounds against different colon cancer cell lines. Compounds were screened via MTS assay for 48 hrs at varying concentrations. The compounds were screened in four different colon cancer cell lines (HCT116, HT29, SW480, and DLD1) and normalized to DMSO. Data is the mean±SD.

FIGS. 4A-4F depict cytotoxicity of PPA24, PPA27, iHAP1, and NSC49L in CRC and FOLFOX-resistant CRC cells: FOLFOX HCT116 (FIG. 4A), FOLFOX HT29 (FIG. 4B), HT29 (FIG. 4C), HCT116 (FIG. 4D), SW480 (FIG. 4E), and DLD1 (FIG. 4F).

FIG. 5 depicts IC₅₀ Values of PPA24, PPA27, iHPA1, and NSC49L in CRC cells. Data obtained from MTS assays using concentrations from 0.5-50 μM. Data is the mean±SD.

FIG. 6 depicts PP2A activity of PPA24, PPA27, iHPA1, and NSC49L. PP2A activity was measured via Malachite green assay using recombinant PP2Ac protein and a threonine phosphopeptide as the substrate. The compounds were treated at concentrations of 250 and 500 nM. The change in color was measured by spectrophotometer at 630 nm. Data is the mean±SD. Analysis was completed using GraphPad Prism 7.0 software.

FIGS. 7A-7B depict measurement of apoptosis in CRC cells after treatment with PPA24, PPA27, iHPA1, and NSC49L. All the cells were treated with PPA-24, PPA-27, iHAP1, and NSC49L at concentrations of 2.5, 5, and 10 M for 24 hrs. FIG. 7A depicts apoptosis measured through Caspase 3/7 and 7-AAD staining. FIG. 7B depicts apoptosis measured through Annexin V and 7-AAD staining. Apoptosis measurement was obtained using a Muse Cell Analyzer.

FIGS. 8A-8C depict cell cycle analysis of PPA24 and PPA27 in CRC cells. All cells were serum starved for 24 hrs. before treatment. FIG. 8A depicts flow cytometry analysis of cell cycle arrest in HCT116 treated with PPA-24 and PPA-27 at 2.5 and 5 M for 30 hrs. Analysis was completed using FCS Express 7. FIG. 8B depicts graphical representation of the cell distribution in the cell cycle phases. FIG. 8C depicts western blot analysis of PPA-24 and PPA-27 treated HCT116 and HT29 cells for cyclin B1 and cyclin E1 protein expression. After serum starvation the cells were treated with PPA-24 or PPA-27 at 2.5 and 5 M for 24 hrs with DMSO as the control. GAPDH was used as the loading control.

FIG. 9 depicts the results from PPA24 screened at 10 μM in the NCI-60 panel of cancer cell lines, indicating an ability to decrease cell viability in most but not all cancer cell lines tested. The varying responses per cell line indicates PPA24 having selectivity towards certain cellular characteristics.

FIG. 10 depicts the results from PPA27 screened at 10 μM in the NCI-60 panel of cancer cell lines, indicating a selectivity regarding the cancer cell line in which PPA27 can induce an effect.

FIGS. 11A-11E depict representative docked poses for PPA24 (FIG. 11A), PPA27 (FIG. 11B), iHAP1 (FIG. 11C), and NSC49L (FIG. 11D) compounds inside of a known pocket of the PP2A-PTPA complex (PDB ID 4LAC). FIG. 11E shows the binding energies of the four compounds.

FIGS. 12A-12E depict surface plasmon resonance (SPR) analysis of the binding of PPA24, PPA27, and NSC49L to catalytic subunit, PP2ACα. FIG. 12A depicts surface plasmon resonance (SPR) studies of PPA24 binding to PP2ACα. FIG. 12B depicts surface plasmon resonance (SPR) studies of PPA27 binding to PP2ACα. FIG. 12C depicts the dissociation constant (K_{D}) of PPA24 binding to PP2ACα. FIG. 12D depicts the dissociation constant (K_{D}) of PPA27 binding to PP2ACα. FIG. 12E depicts surface plasmon resonance (SPR) studies of 100 μM of NSC49L binding to PP2ACα.

FIG. 13 depicts analysis of Oxidative Stress initiated by PPA24 in CRC. HCT116 cells were treated with PPA24 at 2.5 or 5 μM for 3,6, or 24 h before measuring oxidative stress. ROS (+) cells are indicated as M2. ROS (−) cells are indicated by M1 in the above graphs. All cells were analyzed with a Muse Cell Analyzer.

FIGS. 14A-14C depict cell viability screen of PPA24, PPA27, iHAP1, and NSC49L in normal keratinocyte (HaCaT) (FIG. 14A) and epithelial (FHC) (FIG. 14B) cells, where the compounds were able to showcase selectivity to cancerous cells vs. noncancerous cells. FIG. 14C depicts IC₅₀ concentrations of the compounds in the cells. Analyses were done using GraphPad Prism 7.0 Software.

FIGS. 15A-15B depict cell viability data measuring the combination index of PPA24 with Gemcitabine in HCT116. FIG. 15A depicts a heatmap of cell viability for HCT116 cells treated for 48 hrs with PPA24 and Gemcitabine in a checkerboard assay. FIG. 15B depicts combination index (CI) analysis of PPA24 with Gemcitabine in HCT116 cells, showing synergy occurring with this combination. The CI analysis was completed using SiCoDEA.

FIGS. 16A-16D depict SwissADME evaluation of the pharmacological properties of PPA24 (FIG. 16A), PPA27 (FIG. 16B), iHAP1 (FIG. 16C), and NSC49L (FIG. 16D). FIG. 16A depicts the favorable ADME parameters, pharmacokinetic properties, and druglike nature of PPA24. PPA24 is a salt that has been found to be readily water soluble, as opposed to what is predicted by SwissADME.

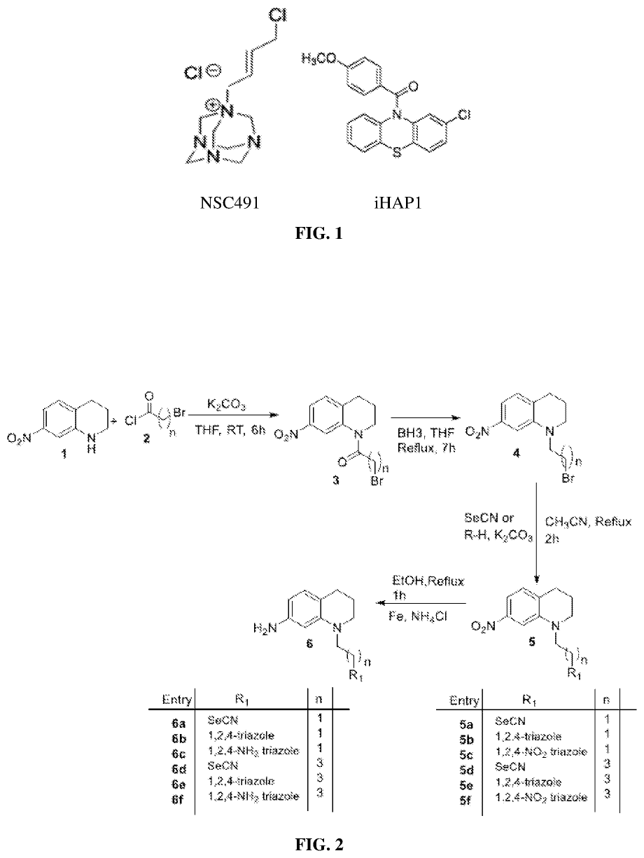

### DEFINITIONS

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched hydrocarbon group having a specified range of carbon atoms (e.g., a “C₁₋₁₆ alkyl” can have from 1 to 16 carbon atoms). In some embodiments, an alkyl group has 1 to 9 carbon atoms (“C₁₋₉ alkyl”). An alkyl group can be saturated or unsaturated, i.e., an alkenyl or alkynyl group as defined herein. Unless specified to the contrary, an “alkyl” group includes both saturated alkyl groups and unsaturated alkyl groups.

In some embodiments, an alkyl group has 1 to 8 carbon atoms (“C₁₋ₛ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”).

In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “alkylenyl” and “alkylene” refers to a divalent radical of a straight-chain, cyclic, or branched saturated hydrocarbon group having a specified range of carbon atoms (e.g., a “C₁₋₁₆ alkyl” can have from 1 to 16 carbon atoms). An example of alkylenyl is a methylene (—CH₂—). An alkylenyl can be substituted as described above for an alkyl.

The term “haloalkyl” is a substituted alkyl group, wherein one or more of the hydrogen atoms are independently replaced by a halogen, e.g., fluoro, bromo, chloro, or iodo.

In some embodiments, the haloalkyl moiety has 1 to 8 carbon atoms (“C₁₋₃ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “hydroxyalkyl” is a substituted alkyl group, wherein one or more of the hydrogen atoms are independently replaced by a hydroxyl. In some embodiments, the hydroxyalkyl moiety has 1 to 8 carbon atoms (“C₁₋ₛ hydroxyalkyl”). In some embodiments, the hydroxyalkyl moiety has 1 to 6 carbon atoms (“C₁₋₆ hydroxyalkyl”). In some embodiments, the hydroxyalkyl moiety has 1 to 4 carbon atoms (“C₁₋₄ hydroxyalkyl”). In some embodiments, the hydroxyalkyl moiety has 1 to 3 carbon atoms (“C₁₋₃ hydroxyalkyl”).

In some embodiments, the hydroxyalkyl moiety has 1 to 2 carbon atoms (“C₁₋₂ hydroxyalkyl”).

The term “alkoxy” refers to an alkyl group, as defined herein, appended to the parent molecular moiety through an oxygen atom. In some embodiments, the alkoxy moiety has 1 to 8 carbon atoms (“C₁₋ₛ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”).

Representative examples of alkoxy include, but are not limited to, methoxy, ethoxy, propoxy, 2-propoxy, butoxy and tert-butoxy.

The term “haloalkoxy” refers to a haloalkyl group, as defined herein, appended to the parent molecular moiety through an oxygen atom. In some embodiments, the alkoxy moiety has 1 to 8 carbon atoms (“C₁₋ₛ haloalkoxy”). In some embodiments, the alkoxy moiety has 1 to 6 carbon atoms (“C₁₋₆ haloalkoxy”). In some embodiments, the alkoxy moiety has 1 to 4 carbon atoms (“C₁₋₄ haloalkoxy”). In some embodiments, the alkoxy moiety has 1 to 3 carbon atoms (“C₁₋₃ haloalkoxy”). In some embodiments, the alkoxy moiety has 1 to 2 carbon atoms (“C₁₋₂ haloalkoxy”). Representative examples of haloalkoxy include, but are not limited to, difluoromethoxy, trifluoromethoxy, and 2,2,2-trifluoroethoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋ₛ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”). By way of example, a C₃alkoxyC₃alkyl group includes, but is not limited to, the groups having the formula: —CH₂CH₂CH₂OCH₂CH₂CH₃, —CH₂CH₂CH₂OCH(CH₃)₂, or —CH(CH₃)CH₂OCH(CH₃)₂, The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and lor more heteroatoms within the parent chain (“heteroC₁₋₁₈ alkyl”). In some embodiments, a heteroalkyl group is a saturated group having 1 to 16 carbon atoms and/or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and lor more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃alkyl”). In some embodiments, a heteroalkyl group is a saturated group having 1 to 2 carbon atoms and 1 heteroatom within the parent chain (“heteroC₁₋₂alkyl”). In some embodiments, a heteroalkyl group is a saturated group having 1 carbon atom and 1 heteroatom (“heteroC₁alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀alkyl. In certain embodiments, the heteroalkyl group is a substituted heteroC₁₋₂₀alkyl. In certain embodiments, the heteroalkyl group is an unsubstituted heteroC₁₋₁₀alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”).

In some embodiments, an alkenyl group has 2 to 6 carbon atoms (“C₂₋₆alkenyl”). In some embodiments, an alkenyl group has 2 to 5 carbon atoms (“C₂₋₅alkenyl”). In some embodiments, an alkenyl group has 2 to 4 carbon atoms (“C₂₋₄alkenyl”). In some embodiments, an alkenyl group has 2 to 3 carbon atoms (“C₂₋₃alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

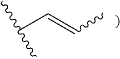

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀alkenyl”). In some embodiments, a heteroalkenyl group has 2 to 9 carbon atoms at least one double bond, and 1 or more heteroatoms within the parent chain (“heteroC₂₋₉alkenyl”).

In some embodiments, a heteroalkenyl group has 2 to 8 carbon atoms, at least one double bond, and 1 or more heteroatoms within the parent chain (“heteroC₂₋₈alkenyl”). In some embodiments, a heteroalkenyl group has 2 to 7 carbon atoms, at least one double bond, and 1 or more heteroatoms within the parent chain (“heteroC₂₋₇alkenyl”). In some embodiments, a heteroalkenyl group has 2 to 6 carbon atoms, at least one double bond, and 1 or more heteroatoms within the parent chain (“heteroC₂₋₆alkenyl”). In some embodiments, a heteroalkenyl group has 2 to 5 carbon atoms, at least one double bond, and 1 or 2 heteroatoms within the parent chain (“heteroC₂₋₅alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃alkenyl”). In some embodiments, a heteroalkenyl group has 2 to 6 carbon atoms, at least one double bond, and 1 or 2 heteroatoms within the parent chain (“heteroC₂₋₆alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀alkenyl. In certain embodiments, the heteroalkenyl group is a substituted heteroC₂₋₁₀alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀alkynyl”). In some embodiments, an alkynyl group has 2 to 9 carbon atoms (“C₂₋₉alkynyl”). In some embodiments, an alkynyl group has 2 to 8 carbon atoms (“C₂₋₈alkynyl”). In some embodiments, an alkynyl group has 2 to 7 carbon atoms (“C₂₋₇ alkynyl”). In some embodiments, an alkynyl group has 2 to 6 carbon atoms (“C₂₋₆alkynyl”).

In some embodiments, an alkynyl group has 2 to 5 carbon atoms (“C₂₋₅alkynyl”). In some embodiments, an alkynyl group has 2 to 4 carbon atoms (“C₂₋₄alkynyl”). In some embodiments, an alkynyl group has 2 to 3 carbon atoms (“C₂₋₃alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀alkynyl”). In some embodiments, a heteroalkynyl group has 2 to 9 carbon atoms, at least one triple bond, and lor more heteroatoms within the parent chain (“heteroC₂₋₉alkynyl”). In some embodiments, a heteroalkynyl group has 2 to 8 carbon atoms, at least one triple bond, and lor more heteroatoms within the parent chain (“heteroC₂₋₈alkynyl”). In some embodiments, a heteroalkynyl group has 2 to 7 carbon atoms, at least one triple bond, and 1 or more heteroatoms within the parent chain (“heteroC₂₋₇alkynyl”). In some embodiments, a heteroalkynyl group has 2 to 6 carbon atoms, at least one triple bond, and 1 or more heteroatoms within the parent chain (“heteroC₂₋₆alkynyl”). In some embodiments, a heteroalkynyl group has 2 to 5 carbon atoms, at least one triple bond, and 1 or 2 heteroatoms within the parent chain (“heteroC₂₋₅alkynyl”). In some embodiments, a heteroalkynyl group has 2 to 4 carbon atoms, at least one triple bond, and 1 or 2 heteroatoms within the parent chain (“heteroC₂₋₄alkynyl”). In some embodiments, a heteroalkynyl group has 2 to 3 carbon atoms, at least one triple bond, and 1 heteroatom within the parent chain (“heteroC₂₋₃alkynyl”). In some embodiments, a heteroalkynyl group has 2 to 6 carbon atoms, at least one triple bond, and 1 or 2 heteroatoms within the parent chain (“heteroC₂₋₆alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀alkynyl. In certain embodiments, the heteroalkynyl group is a substituted heteroC₂₋₁₀alkynyl.

The term “carbocyclyl,” “cycloalkyl,” or “carbocyclic” refers to a radical of a non-aromatic cyclic hydrocarbon group having from 3 to 14 ring carbon atoms (“C₃₋₁₄carbocyclyl”) and zero heteroatoms in the non-aromatic ring system. In some embodiments, a carbocyclyl group has 3 to 10 ring carbon atoms (“C₃₋₁₀carbocyclyl”). In some embodiments, a carbocyclyl group has 3 to 8 ring carbon atoms (“C₃₋₈carbocyclyl”). In some embodiments, a carbocyclyl group has 3 to 7 ring carbon atoms (“C₃₋₇carbocyclyl”). In some embodiments, a carbocyclyl group has 3 to 6 ring carbon atoms (“C₃₋₆carbocyclyl”). In some embodiments, a carbocyclyl group has 4 to 6 ring carbon atoms (“C₄₋₆carbocyclyl”). In some embodiments, a carbocyclyl group has 5 to 6 ring carbon atoms (“C₅₋₆carbocyclyl”). In some embodiments, a carbocyclyl group has 5 to 10 ring carbon atoms (“C₅₋₁₀carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like.

Exemplary C₃₋₈carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄cycloalkyl”). In some embodiments, a cycloalkyl group has 3 to 10 ring carbon atoms (“C₃₋₁₀cycloalkyl”). In some embodiments, a cycloalkyl group has 3 to 8 ring carbon atoms (“C₃₋₈cycloalkyl”). In some embodiments, a cycloalkyl group has 3 to 6 ring carbon atoms (“C₃₋₆cycloalkyl”). In some embodiments, a cycloalkyl group has 4 to 6 ring carbon atoms (“C₄₋₆cycloalkyl”). In some embodiments, a cycloalkyl group has 5 to 6 ring carbon atoms (“C₅₋₆cycloalkyl”). In some embodiments, a cycloalkyl group has 5 to 10 ring carbon atoms (“C₅₋₁₀cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits.

A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, aziridinyl, oxiranyl, and thiiranyl. Exemplary 4-membered heterocyclyl groups containing 1 heteroatom include, without limitation, azetidinyl, oxetanyl, and thietanyl.

Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄aryl”). In some embodiments, an aryl group has 6 ring carbon atoms (“C₆aryl”; e.g., phenyl). In some embodiments, an aryl group has 10 ring carbon atoms (“C₁₀aryl”; e.g., naphthyl such as 1-naphthyl and 2-naphthyl). In some embodiments, an aryl group has 14 ring carbon atoms (“C₁₄aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur.

In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

In general, the inclusion of the prefix “alk” in front of a substituent name indicates there is an alkyl group (as defined herein) connecting the named substitutent with the rest of the compound. For example, “alkaryl” (which is a subset of alkyl) refers to an alkyl group substituted by an aryl group, wherein the point of attachment is on the alkyl moiety and “alkheteroaryl” (which is a subset of “alkyl”) refers to an alkyl group substituted by a heteroaryl group, wherein the point of attachment is on the alkyl moiety. The number of carbons atoms may be specified in the alkyl chain, the named substituent, or both. For example, C₁₋₂alkC₆aryl refers to a phenyl ring (which may be substituted) connected via a 1-2 carbon alkylene group.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR—, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR—, —OC(═NR^{bb})R^{aa}, —OC(═NR^{bb})OR—, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₂, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀alkyl, C₁₋₁₀perhaloalkyl, C₂₋₁₀ alkenyl, C₂₋₁₀alkynyl, heteroC₁₋₁₀alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion; or two geminal hydrogens on a carbon atom are replaced with the group ═O, ═S, ═NN(R^{bb})₂, ═NNR^{bb}C(═O)R—, ═NNR^{bb}C(═O)OR^{aa}, ═NNR^{bb}S(═O)₂R^{aa}, ═NR^{bb} or =NOR^{cc}; each instance of R^{aa} is, independently, selected from C₁₋₁₀alkyl, C₁₋₁₀perhaloalkyl, C₂₋₁₀alkenyl, C₂₋₁₀ alkynyl, heteroC₁₋₁₀alkyl, heteroC₂₋₁₀ alkenyl, heteroC₂₋₁₀alkynyl, C₃₋₁₀ carbocyclyl, 3-14 membered heterocyclyl, C₆₋₁₄aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀alkenyl, C₂₋₁₀alkynyl, heteroC₁₋₁₀alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion; each instance of R^{cc} is, independently, selected from hydrogen, C₁₋₁₀alkyl, C₁₋₁₀perhaloalkyl, C₂₋₁₀alkenyl, C₂₋₁₀alkynyl, heteroC₁₋₁₀ alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R)₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₈ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X is a counterion; each instance of R^{ee} is, independently, selected from C₁₋₆ alkyl, C₁₋₆perhaloalkyl, C₂₋₆alkenyl, C₂₋₆alkynyl, heteroC₁₋₆ alkyl, heteroC₂₋₆alkenyl, membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{f} is, independently, selected from hydrogen, C₁₋₆ alkyl, C₁₋₈ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C_{1- 6} alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₈ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃, —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆perhaloalkyl, C₂₋₆alkenyl, C₂₋₆alkynyl, heteroC₁₋₆ alkyl, heteroC₂₋₆alkenyl, heteroC₂₋₆alkynyl, C₃₋₁₀carbocyclyl, C₆₋₁₀aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R—, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{aa})₂, —OP(R^{aa})₃⁺X⁻, —OP(OR^{aa})₂, —OP(OR^{aa})₃⁺X⁻, —OP(═O)(R^{aa})₂, —OP(═O)(OR^{aa})₂, and —OP(═O)(N(R^{bb})₂)₂, wherein X⁻, R^{aa}, R^{bb} and R^{aa} are as defined herein.

The term “amino” refers to the group —NH₂. The term “substituted amino,” by extension, refers to a monosubstituted amino, a disubstituted amino, or a trisubstituted amino.

In certain embodiments, the “substituted amino” is a monosubstituted amino or a disubstituted ammino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R—, —NHCO₂R^{aa}, —NHC(═O)N(R^{bb})₂, —NHC(═NR^{bb})N(R^{bb})₂, —NHSO₂R^{aa}, —NHP(═O)(OR^{aa})₂, and —NHP(═O)(N(R^{bb})₂)₂, wherein R^{aa}, R^{bb}, and R^{aa} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R—, —NR^{bb}CO₂R—, —NR^{bb}C(═O)N(R^{bb})₂, —NR^{bb}C(═NR^{bb})N(R^{bb})₂, —NR^{bb}SO₂R—, —NR^{bb}P(═O)(OR^{aa})₂, and —NR^{bb}P(═O)(N(R^{bb})₂)₂, wherein R^{aa}, R^{bb}, and R^{aa} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₂ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or dialkylamino, mono- or di-heteroalkylamino, mono- or di-arylamino, or mono- or diheteroarylamino; or two R^{X1} groups taken together form a 5-to 6-membered heterocyclic ring.

Exemplary acyl groups include aldehydes (—CHO), carboxylic acids (—CO₂H), ketones, acyl halides, esters, amides, imines, carbonates, carbamates, and ureas. Acyl substituents include, butare not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes(CHO), esters (e.g., —CO₂R^{aa}, —C(═O)SR^{aa}, —C(═S)SR^{aa}), amides (e.g., —C(═O)N(R^{bb})₂, C(═O)NR^{bb}SO₂R^{aa}, C(═S)N(R^{bb})₂, and imines (e.g., —C(═NR^{bb})R^{aa}, C(═NR^{bb})OR^{aa}), C(═NR^{bb})N(R^{bb})₂, wherein R^{aa} and R^{bb} are as defined herein.

The term “oxo” refers to the group ═O, and the term “thiooxo” refers to the group ═S.

The term “cyano” refers to the group —CN.

The term “azide” and “azido” refers to the group —N₃.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{aa})₂, —CN, —C(═O)R^{aa}, —C(═O)N(R^{aa})₂, —CO₂R^{aa}, —SO₂R^{aa}, —C(═NR^{bb})R—, —C(═NR^{aa})OR^{aa}, —C(═NR^{cc})N(R^{c})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or a 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

As used herein, a chemical bond depicted: [symbol] represents either a single, double, or triple bond, valency permitting. By way of example,

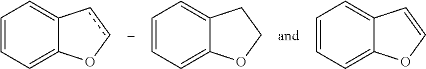

An electron-withdrawing group is a functional group or atom that pulls electron density towards itself, away from other portions of the molecule, e.g., through resonance and/or inductive effects. Exemplary electron-withdrawing groups include F, Cl, Br, I, NO₂, CN, SO₂R, SO₃R, SO₂NR₂, C(O)R^{1a}; C(O)OR, and C(O)NR₂ (wherein R is H or an alkyl, aryl, heteroaryl, cycloalkyl, heterocyclyl group) as well as alkyl group substituted with one or more of those group

An electron-donating group is a functional group or atom that pushes electron density away from itself, towards other portions of the molecule, e.g., through resonance and/or inductive effects. Exemplary electron-donating groups include unsubstituted alkyl or aryl groups, OR and N(R)₂ and alkyl groups substituted with one or more OR and N(R)₂ groups.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Some compounds disclosed herein may exist as one or more tautomers. Tautomers are interconvertible structural isomers that differ in the position of one or more protons or other labile atom. By way of example:

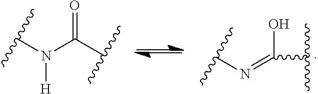

The prevalence of one tautomeric form over another will depend both on the specific chemical compound as well as its local chemical environment. Unless specified to the contrary, the depiction of one tautomeric form is inclusive of all possible tautomeric forms.

Unless stated to the contrary, a substituent drawn without explicitly specifying the point of attachment indicates that the substituent may be attached at any possible atom. For example, in a benzofuran depicted as:

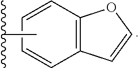

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Compounds disclosed herein may be provided in the form of pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the present disclosure is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the present disclosure are administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for agonism of protein phosphatase 2A. In certain embodiments, a therapeutically effective amount is an amount sufficient for treating cancer. In certain embodiments, a therapeutically effective amount is an amount sufficient for agonism of protein phosphatase 2A and treating cancer.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for agonism of protein phosphatase 2A. In certain embodiments, a prophylactically effective amount is an amount sufficient for treating cancer. In certain embodiments, a prophylactically effective amount is an amount sufficient for agonism of protein phosphatase 2A and treating cancer.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

As used herein “CRC” refers to colorectal cancer.

As used herein “FOLFOX” refers to a therapeutic regimen that includes administration of 5-fluorouracil (“5-FU”), folinic acid, and oxaliplatin.

The term “linker” refers to a bond or a divalent chemical moiety that is bonded to (i.e., that connects) two separate monovalent chemical moieties (e.g., Het and X in Formula (I)).

### DETAILED DESCRIPTION

### Compounds

In one aspect, the present disclosure provides a compound of any of the formulae herein, or salt thereof.

In one aspect, the present disclosure provides a compound of Formula (I):

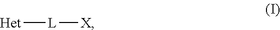

or a pharmaceutically acceptable salt thereof, wherein:
Het represents a heterocyclyl group;
L represents a linker; and
X represents a positively charged, bulky group.

### X

### Formula (II)

In some embodiments, X is of Formula (II):

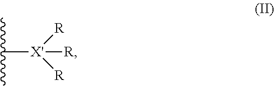

wherein:
X′ is N or C; and
each instance of R is independently selected from H, C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl, wherein any two or more instances of R can together form a ring;
wherein when X′ is C, then at least one instance of R is a C₁₋₁₂ heteroalkyl group having at least one quaternary nitrogen atom.

As generally described herein, X′ is N or C.

In some embodiments, X′ is N. In some embodiments, X′ is C. In some embodiments, X′ is C and at least one instance of R is a C₁₋₁₂ heteroalkyl group having at least one quaternary nitrogen atom.

As generally described herein, each instance of R is independently selected from H, C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl, wherein any two or more instances of R can together form a ring.

In some embodiments, no instance of R is hydrogen.

In some embodiments, each instance of R is independently selected from C₁₋₁₂ alkyl and C₁₋₁₂ heteroalkyl, wherein any two or more instances of R can together form a ring. In some embodiments, two instances of R together form a ring. In some embodiments, three instances of R together form a ring (i.e., a ring system). In some embodiments, two or more instances of R together form a carbocyclic ring. In some embodiments, two or more instances of R together form a heterocyclic ring.

### Formula (II-a)

In some embodiments, X is of Formula (II-a):

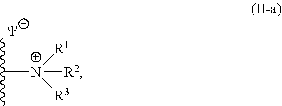

wherein:
R¹ is C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl;
R² is C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl;
R³ is C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl; and
Ψ represents an anionic group;
wherein any two or more of R¹, R², and R³ may together form a ring.

As generally described herein, R¹ is C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl; R² is C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl; and R³ is C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl; wherein any two or more of R¹, R², and R³ may together form a ring.

In some embodiments, R¹ is C₁₋₁₂ alkyl or C₁₋₁₂ heteroalkyl. In some embodiments, R¹ is C₁₋₆ alkyl. In some embodiments, R¹ is C₁₋₃ alkyl. In some embodiments, R¹ is C₁₋₆ heteroalkyl. In some embodiments, R¹ is C₁₋₃ heteroalkyl.

In some embodiments, R² is C₁₋₁₂ alkyl or C₁₋₁₂ heteroalkyl. In some embodiments, R² is C₁₋₆ alkyl. In some embodiments, R² is C₁₋₃ alkyl. In some embodiments, R² is C₁₋₆ heteroalkyl. In some embodiments, R² is C₁₋₃ heteroalkyl.

In some embodiments, R³ is C₁₋₁₂ alkyl or C₁₋₁₂ heteroalkyl. In some embodiments, R³ is C₁₋₆ alkyl. In some embodiments, R³ is C₁₋₃ alkyl. In some embodiments, R³ is C₁₋₆ heteroalkyl. In some embodiments, R³ is C₁₋₃ heteroalkyl.

In some embodiments, R¹ and R² together form a ring. In some embodiments, R¹ and R² together form a heterocyclic ring. In some embodiments, R¹ and R² together form a 5-6 membered heterocyclic ring. In some embodiments, R¹ and R² together form a 5 membered heterocyclic ring. In some embodiments, R¹ and R² together form a 6 membered heterocyclic ring.

In some embodiments, R¹ and R² together form a heterocyclic ring containing at least one quaternary nitrogen atom. In some embodiments, R¹ and R² together form a 5-6 membered heterocyclic ring containing at least one quaternary nitrogen atom. In some embodiments, R¹ and R² together form a 5 membered heterocyclic ring containing at least one quaternary nitrogen atom. In some embodiments, R¹ and R² together form a 6 membered heterocyclic ring containing at least one quaternary nitrogen atom.

In some embodiments, R² and R³ together form a ring. In some embodiments, R² and R³ together form a heterocyclic ring. In some embodiments, R² and R³ together form a 5-6 membered heterocyclic ring. In some embodiments, R² and R³ together form a 5 membered heterocyclic ring. In some embodiments, R² and R³ together form a 6 membered heterocyclic ring.

In some embodiments, R² and R³ together form a heterocyclic ring containing at least one quaternary nitrogen atom. In some embodiments, R² and R³ together form a 5-6 membered heterocyclic ring containing at least one quaternary nitrogen atom. In some embodiments, R² and R³ together form a 5 membered heterocyclic ring containing at least one quaternary nitrogen atom. In some embodiments, R² and R³ together form a 6 membered heterocyclic ring containing at least one quaternary nitrogen atom.

In some embodiments, R¹ and R³ together form a ring. In some embodiments, R¹ and R³ together form a heterocyclic ring. In some embodiments, R¹ and R³ together form a 5-6 membered heterocyclic ring. In some embodiments, R¹ and R³ together form a 5 membered heterocyclic ring. In some embodiments, R¹ and R³ together form a 6 membered heterocyclic ring.

In some embodiments, R¹ and R³ together form a heterocyclic ring containing at least one quaternary nitrogen atom. In some embodiments, R¹ and R³ together form a 5-6 membered heterocyclic ring containing at least one quaternary nitrogen atom. In some embodiments, R¹ and R³ together form a 5 membered heterocyclic ring containing at least one quaternary nitrogen atom. In some embodiments, R¹ and R³ together form a 6 membered heterocyclic ring containing at least one quaternary nitrogen atom.

As generally described herein, Ψ represents an anionic group.

In some embodiments, Ψ is covalently bound to the compound. In some embodiments, Ψ is selected from COOH, SO₃H, and PO₃H₂. In some embodiments, Ψ is selected from COO—, SO₃, and PO₃H—.

In some embodiments, Ψ is COOH. In some embodiments, Ψ is COO—.

In some embodiments, Ψ is not covalently bond to the compound. In some embodiments, Ψ is selected from halide, carboxylate, sulfate, nitrate, and phosphate.

In some embodiments, Ψ is halide. In some embodiments, Ψ is chloride or bromide.

In some embodiments, Ψ is bromide.

### Formula (II-b)

In some embodiments, X is of Formula (II-b):

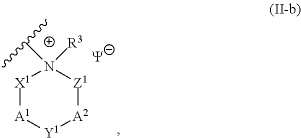

wherein:
X¹ is null, C₁₋₈ alkylene, or C₁₋₈ heteroalkylene;
Y¹ is null, C₁₋₈ alkylene, or C₁₋₈ heteroalkylene;
Z¹ is null, C₁₋₈ alkylene, or C₁₋₈ heteroalkylene;
A¹ is null, CHR⁴ or NR⁵;
A² is null, CHR⁶ or NR⁷;
R³ is C₁₋₈ alkyl or C₁₋₈ heteroalkyl;
R⁴ is H, C₁₋₈ alkyl, or C₁₋₈ heteroalkyl;
R⁵ is H, C₁₋₈ alkyl, or C₁₋₈ heteroalkyl;
R⁶ is H, C₁₋₈ alkyl, or C₁₋₈ heteroalkyl;
R⁷ is H, C₁₋₈ alkyl, or C₁₋₈ heteroalkyl; and
Ψ represents an anionic group;
wherein any two or more of X¹, Y¹, Z¹, R³, R⁴, R⁵, R⁶, or R⁷ can together form a ring; with the proviso that the compound includes at least one of X¹, Y¹, Z¹, and at least one of A¹ or A².

As generally described herein, X¹ is null, C₁₋₈ alkylene, or C₁₋₈ heteroalkylene; Y¹ is null, C₁₋₈ alkylene, or C₁₋₈ heteroalkylene; Z¹ is null, C₁₋₈ alkylene, or C₁₋₈ heteroalkylene; A¹ is null, CHR⁴ or NR⁵; A² is null, CHR⁶ or NR⁷; R⁴ is H, C₁₋₈ alkyl, or C₁₋₈ heteroalkyl; R⁵ is H, C₁₋₈ alkyl, or C₁₋₈ heteroalkyl; R⁶ is H, C₁₋₈ alkyl, or C₁₋₈ heteroalkyl; and R⁷ is H, C₁₋₈ alkyl, or C₁₋₈ heteroalkyl; wherein any two or more of X¹, Y¹, Z¹, R³, R⁴, R⁵, R⁶, or R⁷ can together form a ring.

In some embodiments, X¹ is null, C₁₋₈ alkylene, or C₁₋₈ heteroalkylene. In some embodiments, X¹ is null. In some embodiments, X¹ is C₁₋₈ alkylene. In some embodiments, X¹ is C₁₋₄ alkylene. In some embodiments, X¹ is C₁₋₂ alkylene. In some embodiments, X¹ is methylene or ethylene. In some embodiments, X¹ is C₁₋₈ heteroalkylene. In some embodiments, X¹ is C₁₋₄ heteroalkylene. In some embodiments, X¹ is C₁₋₂ heteroalkylene.

In some embodiments, Y¹ is null, C₁₋₈ alkylene, or C₁₋₈ heteroalkylene. In some embodiments, Y¹ is null. In some embodiments, Y¹ is C₁₋₈ alkylene. In some embodiments, Y¹ is C₁₋₄ alkylene. In some embodiments, Y¹ is C₁₋₂ alkylene. In some embodiments, Y¹ is methylene or ethylene. In some embodiments, Y¹ is C₁₋₈ heteroalkylene. In some embodiments, Y¹ is C₁₋₄ heteroalkylene. In some embodiments, Y¹ is C₁₋₂ heteroalkylene.

In some embodiments, Z¹ is null, C₁₋₈ alkylene, or C₁₋₈ heteroalkylene. In some embodiments, Z¹ is null. In some embodiments, Z¹ is C₁₋₈ alkylene. In some embodiments, Z¹ is C₁₋₄ alkylene. In some embodiments, Z¹ is C₁₋₂ alkylene. In some embodiments, Z¹ is methylene or ethylene. In some embodiments, Z¹ is C₁₋₈ heteroalkylene. In some embodiments, Z¹ is C₁₋₄ heteroalkylene. In some embodiments, Z¹ is C₁₋₂ heteroalkylene.

In some embodiments, at least one of X¹, Y¹, and Z¹ is methylene. In some embodiments, each of X¹, Y¹, and Z¹ is independently methylene.

In some embodiments, at least one of X¹, Y¹, and Z¹ is ethylene. In some embodiments, each of X¹, Y¹, and Z¹ is independently ethylene.

In some embodiments, A¹ is null, CHR⁴ or NR⁵. In some embodiments, A¹ is null. In some embodiments, A¹ is CHR⁴ or NR⁵. In some embodiments, A¹ is CHR⁴. In some embodiments, A¹ is NR⁵.

In some embodiments, A² is null, CHR⁶ or NR⁷. In some embodiments, A² is null. In some embodiments, A² is CHR⁶ or NR⁷. In some embodiments, A² is CHR⁶. In some embodiments, A² is NR⁷.

In some embodiments, R³ is C₁₋₈ alkyl. In some embodiments, R³ is C₁₋₄ alkyl. In some embodiments, R³ is C₁₋₂ alkyl. In some embodiments, R³ is C₁₋₈ heteroalkyl. In some embodiments, R³ is C₁₋₄ heteroalkyl. In some embodiments, R³ is C₁₋₂ heteroalkyl.

In some embodiments, R⁴ is H, C₁₋₈ alkyl, or C₁₋₈ heteroalkyl. In some embodiments, R⁴ is H. In some embodiments, R⁴ is C₁₋₄ alkyl. In some embodiments, R⁴ is C₁₋₄ heteroalkyl.

In some embodiments, R⁵ is H, C₁₋₈ alkyl, or C₁₋₈ heteroalkyl. In some embodiments, R⁵ is H. In some embodiments, R⁵ is C₁₋₄ alkyl. In some embodiments, R⁵ is C₁₋₄ heteroalkyl.

In some embodiments, R⁶ is H, C₁₋₈ alkyl, or C₁₋₈ heteroalkyl. In some embodiments, R⁶ is H. In some embodiments, R⁶ is C₁₋₄ alkyl. In some embodiments, R⁶ is C₁₋₄ heteroalkyl.

In some embodiments, R⁷ is H, C₁₋₈ alkyl, or C₁₋₈ heteroalkyl. In some embodiments, R⁷ is H. In some embodiments, R⁷ is C₁₋₄ alkyl. In some embodiments, R⁷ is C₁₋₄ heteroalkyl.

In some embodiments, any two or more of X¹, Y¹, Z¹, R³, R⁴, R⁵, R⁶, or R⁷ together form a ring. In some embodiments, any two or more of R³, R⁴, R⁵, R⁶, or R⁷ together form a ring.

In some embodiments, R⁴ and R⁶ together form a ring. In some embodiments, R⁵ and R⁶ together form a ring. In some embodiments, R⁴ and R⁷ together form a ring. In some embodiments, R⁵ and R⁷ together form a ring.

In some embodiments, R⁴ and R⁶ together form a C₁₋₄ alkylene group. In some embodiments, R⁵ and R⁶ together form a C₁₋₄ alkylene group. In some embodiments, R⁴ and R⁷ together form a C₁₋₄ alkylene group. In some embodiments, R⁵ and R⁷ together form a C₁₋₄ alkylene group.

In some embodiments, Y¹ and A² are null.

As generally described herein, Ψ represents an anionic group.

In some embodiments, Ψ is covalently bound to the compound. In some embodiments, Ψ is selected from COOH, SO₃H, and PO₃H₂. In some embodiments, Ψ is selected from COO—, SO₃, and PO₃H—.

In some embodiments, Ψ is COOH. In some embodiments, Ψ is COO—.

In some embodiments, Ψ is not covalently bond to the compound. In some embodiments, Ψ is selected from halide, carboxylate, sulfate, nitrate, and phosphate.

In some embodiments, Ψ is halide. In some embodiments, Ψ is chloride or bromide.

In some embodiments, Ψ is bromide.

### Formula (II-c)

In some embodiments, X is of Formula (II-c):

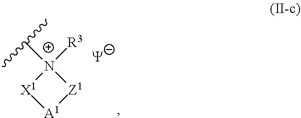

wherein:
X¹ is C₁₋₄ alkylene, preferably ethylene;
Z¹ is C₁₋₄ alkylene, preferably ethylene;
A¹ is null, CHR⁴ or NR⁵;
R³ is C₁₋₈ alkyl or C₁₋₈ heteroalkyl;
R⁴ is H, C₁₋₈ alkyl, or C₁₋₈ heteroalkyl;
R⁵ is H, C₁₋₈ alkyl, or C₁₋₈ heteroalkyl; and
Ψ represents an anionic group;
wherein any two or more of X¹, Z¹, R³, R⁴, or R⁵ can together form a ring.

As generally described herein, X¹ is C₁₋₄ alkylene, preferably ethylene; Z¹ is C₁₋₄ alkylene, preferably ethylene; A¹ is null, CHR⁴ or NR⁵; R³ is C₁₋₈ alkyl or C₁₋₈ heteroalkyl; R⁴ is H, C₁₋₈ alkyl, or C₁₋₈ heteroalkyl; and R⁵ is H, C₁₋₈ alkyl, or C₁₋₈ heteroalkyl; wherein any two or more of X¹, Z¹, R³, R⁴, or R⁵ can together form a ring.

In some embodiments, X¹ is C₁₋₂ alkylene. In some embodiments, X¹ is methylene or ethylene. In some embodiments, X¹ is ethylene.

In some embodiments, Z¹ is C₁₋₂ alkylene. In some embodiments, Z¹ is methylene or ethylene. In some embodiments, Z¹ is ethylene.

In some embodiments, A¹ is null. In some embodiments, A¹ is CHR⁴ or NR⁵. In some embodiments, A¹ is CHR⁴. In some embodiments, A¹ is NR⁵.

In some embodiments, R³ is C₁₋₈ alkyl. In some embodiments, R³ is C₁₋₄ alkyl. In some embodiments, R³ is C₁₋₂ alkyl. In some embodiments, R³ is C₁₋₈ heteroalkyl. In some embodiments, R³ is C₁₋₄ heteroalkyl. In some embodiments, R³ is C₁₋₂ heteroalkyl.

In some embodiments, R⁴ is C₁₋₄ alkyl. In some embodiments, R⁴ is C₁₋₄ heteroalkyl.

In some embodiments, R⁵ is C₁₋₄ alkyl. In some embodiments, R⁵ is C₁₋₄ heteroalkyl.

In some embodiments, any two or more of X¹, Z¹, R³, R⁴, or R⁵ together form a ring.

In some embodiments, any two or more of R³, R⁴, or R⁵ together form a ring.

In some embodiments, R³ and R⁴ together form a ring. In some embodiments, R³ and R⁴ together form a C₁₋₄ alkylene group.

In some embodiments, R³ and R⁵ together form a ring. In some embodiments, R³ and R⁵ together form a C₁₋₄ alkylene group.

In some embodiments, R⁴ and R⁵ together form a ring. In some embodiments, R⁴ and R⁵ together form a C₁₋₄ alkylene group.

As generally described herein, Ψ represents an anionic group.

In some embodiments, Ψ is covalently bound to the compound. In some embodiments, Ψ is selected from COOH, SO₃H, and PO₃H₂. In some embodiments, Ψ is selected from COO—, SO₃, and PO₃H—.

In some embodiments, Ψ is COOH. In some embodiments, Ψ is COO—.

In some embodiments, Ψ is not covalently bond to the compound. In some embodiments, Ψ is selected from halide, carboxylate, sulfate, nitrate, and phosphate.

In some embodiments, Ψ is halide. In some embodiments, Ψ is chloride or bromide.

In some embodiments, Ψ is bromide.

### Formula (II-d)

In some embodiments, X is of Formula (II-d):

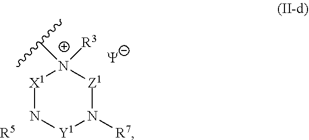

wherein:
X¹ is C₁₋₈ alkylene;
Y¹ is C₁₋₈ alkylene;
Z¹ is C₁₋₈ alkylene;
R³ is C₁₋₈ alkyl or C₁₋₈ heteroalkyl;
R⁵ is H, C₁₋₈ alkyl, or C₁₋₈ heteroalkyl;
R⁷ is H, C₁₋₈ alkyl, or C₁₋₈ heteroalkyl; and
Ψ represents an anionic group;
wherein any two or more of X¹, Y¹, Z¹, R³, R⁵, and R⁷ can together form a ring.

As generally described herein, X¹ is C₁₋₈ alkylene; Y¹ is C₁₋₈ alkylene; Z¹ is C₁₋₈ alkylene; R³ is C₁₋₈ alkyl or C₁₋₈ heteroalkyl; R⁵ is H, C₁₋₈ alkyl, or C₁₋₈ heteroalkyl; and R⁷ is H, C₁₋₈ alkyl, or C₁₋₈ heteroalkyl; wherein any two or more of X¹, Y¹, Z¹, R³, R⁵, and R⁷ can together form a ring.

In some embodiments, X¹ is C₁₋₄ alkylene. In some embodiments, X¹ is C₁₋₄ alkylene, preferably ethylene. In some embodiments, X¹ is C₁₋₂ alkylene. In some embodiments, X¹ is methylene or ethylene. In some embodiments, X¹ is ethylene.

In some embodiments, Y¹ is C₁₋₄ alkylene. In some embodiments, Y¹ is C₁₋₄ alkylene, preferably ethylene. In some embodiments, Y¹ is C₁₋₂ alkylene. In some embodiments, Y¹ is methylene or ethylene. In some embodiments, Y¹ is ethylene.

In some embodiments, Z¹ is C₁₋₄ alkylene. In some embodiments, Z¹ is C₁₋₄ alkylene, preferably ethylene. In some embodiments, Z¹ is C₁₋₂ alkylene. In some embodiments, Z¹ is methylene or ethylene. In some embodiments, Z¹ is ethylene.

In some embodiments, X¹ is C₁₋₄ alkylene, preferably ethylene; Y¹ is C₁₋₄ alkylene, preferably ethylene; and Z¹ is C₁₋₄ alkylene, preferably ethylene.

In some embodiments, X¹ is ethylene; Y¹ is ethylene; and Z¹ is ethylene.

In some embodiments, R³ is C₁₋₈ alkyl. In some embodiments, R³ is C₁₋₄ alkyl. In some embodiments, R³ is C₁₋₂ alkyl. In some embodiments, R³ is C₁₋₈ heteroalkyl. In some embodiments, R³ is C₁₋₄ heteroalkyl. In some embodiments, R³ is C₁₋₂ heteroalkyl. In some embodiments, R⁵ is C₁₋₈ alkyl or C₁₋₈ heteroalkyl. In some embodiments, R⁵ is H. In some embodiments, R⁵ is C₁₋₄ alkyl. In some embodiments, R⁵ is C₁₋₄ heteroalkyl.

In some embodiments, R⁷ is C₁₋₈ alkyl or C₁₋₈ heteroalkyl. In some embodiments, R⁷ is H. In some embodiments, R⁷ is C₁₋₄ alkyl. In some embodiments, R⁷ is C₁₋₄ heteroalkyl.

In some embodiments, any two or more of X¹, Y¹, Z¹, R³, R⁵, and R⁷ together form a ring. In some embodiments, any two or more of R³, R⁵, and R⁷ together form a ring.

In some embodiments, R³ and R⁵ together form a ring. In some embodiments, R³ and R⁷ together form a ring. In some embodiments, R⁵ and R⁷ together form a ring.

In some embodiments, R³ and R⁵ together form a C₁₋₄ alkylene group. In some embodiments, R³ and R⁷ together form a C₁₋₄ alkylene group. In some embodiments, R⁵ and R⁷ together form a C₁₋₄ alkylene group.

In some embodiments, R³, R⁵, and R⁷ together form a ring system.

As generally described herein, Ψ represents an anionic group.

In some embodiments, Ψ is covalently bound to the compound. In some embodiments, Ψ is selected from COOH, SO₃H, and PO₃H₂. In some embodiments, Ψ is selected from COO—, SO₃, and PO₃H—.

In some embodiments, Ψ is COOH. In some embodiments, Ψ is COO—.

In some embodiments, Ψ is not covalently bond to the compound. In some embodiments, Ψ is selected from halide, carboxylate, sulfate, nitrate, and phosphate.

In some embodiments, Ψ is halide. In some embodiments, Ψ is chloride or bromide.

In some embodiments, Ψ is bromide.

### Formula (II-e)

In some embodiments, X is of Formula (II-e):

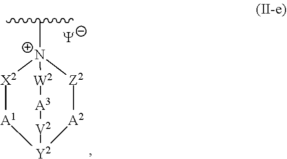

wherein:
   X² is null, C₁₋₈ alkylene, or C₁₋₈ heteroalkylene;
   A¹ is null, CHR⁴ or NR⁵;
wherein A¹ and X² cannot both be null;
   Y² is N, C₁₋ₛ alkylene, or C₁₋₈ heteroalkylene;
   Z² is null, C₁₋₈ alkylene, or C₁₋₈ heteroalkylene;
   A² is null, CHR⁶ or NR⁷;
wherein A² and Z² cannot both be null;
   W² is null, C₁₋₈ alkylene, or C₁₋₈ heteroalkylene;
   V² is null, C₁₋₈ alkylene, or C₁₋₈ heteroalkylene;
   A³ is null, CHR⁸ or NR⁹;
wherein A², W², and V² cannot all be null,
   R⁴ is H, C₁₋₈ alkyl, or C₁₋₈ heteroalkyl;
   R⁵ is H, C₁₋₈ alkyl, or C₁₋₈ heteroalkyl;
   R⁶ is H, C₁₋₈ alkyl, or C₁₋₈ heteroalkyl;
   R⁷ is H, C₁₋₈ alkyl, or C₁₋₈ heteroalkyl;
   R⁸ is H, C₁₋₈ alkyl, or C₁₋₈ heteroalkyl;
   R⁹ is H, C₁₋₈ alkyl, or C₁₋₈ heteroalkyl; and
   Ψ represents an anionic group;
wherein any two or more of R⁴, R⁵, R⁶, R⁷, R⁸, and R⁹ can together form a ring.

As generally described herein, X² is null, C₁₋₈ alkylene, or C₁₋₈ heteroalkylene; A¹ is null, CHR⁴ or NR⁵; wherein A¹ and X² cannot both be null; Y² is N, C₁₋₈ alkylene, or C₁₋₈ heteroalkylene; Z² is null, C₁₋₈ alkylene, or C₁₋₈ heteroalkylene; A² is null, CHR⁶ or NR⁷; wherein A² and Z² cannot both be null; W² is null, C₁₋₈ alkylene, or C₁₋₈ heteroalkylene; V² is null, C₁₋₈ alkylene, or C₁₋₈ heteroalkylene; and A³ is null, CHR⁸ or NR⁹; wherein A², W², and V² cannot all be null.

In some embodiments, X² is null. In some embodiments, X² is C₁₋₄ alkylene. In some embodiments, X² is C₁₋₂ alkylene.

In some embodiments, A¹ is null. In some embodiments, A¹ is CHR⁴. In some embodiments, A¹ is NR⁵.

In some embodiments, Y² is N. In some embodiments, Y² is C₁₋₈ alkylene. In some embodiments, Y² is C₁₋₄ alkylene. In some embodiments, Y² is C₁₋₈ heteroalkylene. In some embodiments, Y² is C₁₋₄ heteroalkylene.

In some embodiments, Z² is null. In some embodiments, Z² is C₁₋₈ alkylene. In some embodiments, Z² is C₁₋₄ alkylene. In some embodiments, Z² is C₁₋₈ heteroalkylene. In some embodiments, Z² is C₁₋₄ heteroalkylene.

In some embodiments, A² is null. In some embodiments, A² is CHR⁶. In some embodiments, A² is NR⁷.

In some embodiments, W² is null. In some embodiments, W² is C₁₋₈ alkylene. In some embodiments, W² is C₁₋₄ alkylene. In some embodiments, W² is C₁₋₈ heteroalkylene. In some embodiments, W² is C₁₋₄ heteroalkylene.

In some embodiments, V² is null. In some embodiments, V² is C₁₋₈ alkylene. In some embodiments, V² is C₁₋₄ alkylene. In some embodiments, V² is C₁₋₈ heteroalkylene. In some embodiments, V² is C₁₋₄ heteroalkylene.

In some embodiments, A³ is null. In some embodiments, A³ is CHR⁸. In some embodiments, A³ is NR⁹.

In some embodiments, A³ and V² are null.

As generally described herein, R⁴ is H, C₁₋₈ alkyl, or C₁₋₈ heteroalkyl; R⁵ is H, C₁₋₈ alkyl, or C₁₋₈ heteroalkyl; R⁶ is H, C₁₋₈ alkyl, or C₁₋₈ heteroalkyl; R⁷ is H, C₁₋₈ alkyl, or C₁₋₈ heteroalkyl; R⁸ is H, C₁₋₈ alkyl, or C₁₋₈ heteroalkyl; and R⁹ is H, C₁₋₈ alkyl, or C₁₋₈ heteroalkyl; wherein any two or more of R⁴, R⁵, R⁶, R⁷, R⁸, and R⁹ can together form a ring.

In some embodiments, R⁴ is H. In some embodiments, R⁴ is C₁₋₈ alkyl. In some embodiments, R⁴ is C₁₋₄ alkyl. In some embodiments, R⁴ is C₁₋₈ heteroalkyl. In some embodiments, R⁴ is C₁₋₄ heteroalkyl.

In some embodiments, R⁵ is H. In some embodiments, R⁵ is C₁₋₈ alkyl. In some embodiments, R⁵ is C₁₋₄ alkyl. In some embodiments, R⁵ is C₁₋₈ heteroalkyl. In some embodiments, R⁵ is C₁₋₄ heteroalkyl.

In some embodiments, R⁶ is H. In some embodiments, R⁶ is C₁₋₈ alkyl. In some embodiments, R⁶ is C₁₋₄ alkyl. In some embodiments, R⁶ is C₁₋₈ heteroalkyl. In some embodiments, R⁶ is C₁₋₄ heteroalkyl.

In some embodiments, R⁷ is H. In some embodiments, R⁷ is C₁₋₈ alkyl. In some embodiments, R⁷ is C₁₋₄ alkyl. In some embodiments, R⁷ is C₁₋₈ heteroalkyl. In some embodiments, R⁷ is C₁₋₄ heteroalkyl.

In some embodiments, R⁸ is H. In some embodiments, R⁸ is C₁₋₈ alkyl. In some embodiments, R⁸ is C₁₋₄ alkyl. In some embodiments, R⁸ is C₁₋₈ heteroalkyl. In some embodiments, R⁸ is C₁₋₄ heteroalkyl.

In some embodiments, R⁹ is H. In some embodiments, R⁹ is C₁₋₈ alkyl. In some embodiments, R⁹ is C₁₋₄ alkyl. In some embodiments, R⁹ is C₁₋₈ heteroalkyl. In some embodiments, R⁹ is C₁₋₄ heteroalkyl.

In some embodiments, any two or more of R⁴, R⁵, R⁶, R⁷, R⁸, and R⁹ together form a ring.

In some embodiments, R⁴ and R⁶ together form a ring. In some embodiments, R⁴ and R⁷ together form a ring. In some embodiments, R⁴ and R⁸ together form a ring. In some embodiments, R⁴ and R⁹ together form a ring. In some embodiments, R⁶ and R⁸ together form a ring. In some embodiments, R⁷ and R⁸ together form a ring. In some embodiments, R⁶ and R⁹ together form a ring.

In some embodiments, R⁵ and R⁶ together form a ring. In some embodiments, R⁵ and R⁷ together form a ring. In some embodiments, R⁵ and R⁸ together form a ring. In some embodiments, R⁵ and R⁹ together form a ring.

In some embodiments, R⁷ and R⁹ together form a ring system. In some embodiments, R⁴, R⁶, and R⁸ together form a ring system.

In some embodiments, R⁵, R⁷, and R⁹ together form a ring system.

In some embodiments, R⁴ and R⁶ together form a C₁₋₄ alkylene group. In some embodiments, R⁴ and R⁷ together form a C₁₋₄ alkylene group. In some embodiments, R⁴ and R⁸ together form a C₁₋₄ alkylene group. In some embodiments, R⁴ and R⁹ together form a C₁₋₄ alkylene group. In some embodiments, R⁶ and R⁸ together form a C₁₋₄ alkylene group. In some embodiments, R⁷ and R⁸ together form a C₁₋₄ alkylene group. In some embodiments, R⁶ and R⁹ together form a C₁₋₄ alkylene group. In some embodiments, R⁷ and R⁹ together form a C₁₋₄ alkylene group.

In some embodiments, R⁵ and R⁶ together form a C₁₋₄ alkylene group. In some embodiments, R⁵ and R⁷ together form a C₁₋₄ alkylene group. In some embodiments, R⁵ and R⁸ together form a C₁₋₄ alkylene group. In some embodiments, R⁵ and R⁹ together form a C₁₋₄ alkylene group.

In some embodiments, R⁴, R⁶, and R⁸ together form a ring system having from 1-8 carbon atoms. In some embodiments, R⁴, R⁶, and R⁸ together form a ring system having from 4-8 carbon atoms.

In some embodiments, R⁵, R⁷, and R⁹ together form a ring system having from 1-8 carbon atoms. In some embodiments, R⁵, R⁷, and R⁹ together form a ring system having from 4-8 carbon atoms.

In some embodiments, Ψ is covalently bound to the compound. In some embodiments, Ψ is selected from COOH, SO₃H, and PO₃H₂. In some embodiments, Ψ is selected from COO—, SO₃, and PO₃H—.

In some embodiments, Ψ is COOH. In some embodiments, Ψ is COO—.

In some embodiments, Ψ is not covalently bond to the compound. In some embodiments, Ψ is selected from halide, carboxylate, sulfate, nitrate, and phosphate.

In some embodiments, Ψ is halide. In some embodiments, Ψ is chloride or bromide. In some embodiments, Ψ is bromide.

### Formula (II-f)

In some embodiments, X is of Formula (II-f):

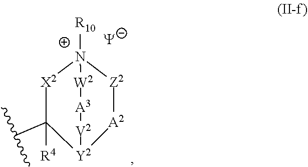

wherein:
   X² is null, C₁₋₈ alkylene, or C₁₋₈ heteroalkylene;
   Y² is N, C₁₋₈ alkylene, or C₁₋₈ heteroalkylene;
   Z² is null, C₁₋₈ alkylene, or C₁₋₈ heteroalkylene;
   A² is null, CHR⁶ or NR⁷;
wherein A² and Z² cannot both be null;
   W² is null, C₁₋₈ alkylene, or C₁₋₈ heteroalkylene;
   V² is null, C₁₋₈ alkylene, or C₁₋₈ heteroalkylene;
   A³ is null, CHR⁸ or NR⁹;
wherein A², W², and V² cannot all be null;
   R⁴ is H, C₁₋₈ alkyl, or C₁₋₈ heteroalkyl;
   R⁶ is H, C₁₋₈ alkyl, or C₁₋₈ heteroalkyl;
   R⁷ is H, C₁₋₈ alkyl, or C₁₋₈ heteroalkyl;
   R⁸ is H, C₁₋₈ alkyl, or C₁₋₈ heteroalkyl;
   R⁹ is H, C₁₋₈ alkyl, or C₁₋₈ heteroalkyl;
   R¹⁰ is C₁₋₈ alkyl or C₁₋₈ heteroalkyl; and
   Ψ represents an anionic group;
wherein any two or more of R⁴, R⁶, R⁷, R⁸, and R⁹ can together form a ring.

As generally described herein, X² is null, C₁₋₈ alkylene, or C₁₋₈ heteroalkylene; Y² is N, C₁₋₈ alkylene, or C₁₋₈ heteroalkylene; Z² is null, C₁₋₈ alkylene, or C₁₋₈ heteroalkylene; A² is null, CHR⁶ or NR⁷; wherein A² and Z² cannot both be null; W² is null, C₁₋₈ alkylene, or C₁₋₈ heteroalkylene; V² is null, C₁₋₈ alkylene, or C₁₋₈ heteroalkylene; A³ is null, CHR⁸ or NR⁹; wherein A², W², and V² cannot all be null;

In some embodiments, X² is null. In some embodiments, X² is C₁₋₄ alkylene. In some embodiments, X² is C₁₋₂ alkylene.

In some embodiments, Y² is N. In some embodiments, Y² is C₁₋₈ alkylene. In some embodiments, Y² is C₁₋₄ alkylene. In some embodiments, Y² is C₁₋₈ heteroalkylene. In some embodiments, Y² is C₁₋₄ heteroalkylene.

In some embodiments, Z² is null. In some embodiments, Z² is C₁₋₈ alkylene. In some embodiments, Z² is C₁₋₄ alkylene. In some embodiments, Z² is C₁₋₈ heteroalkylene. In some embodiments, Z² is C₁₋₄ heteroalkylene.

In some embodiments, A² is null. In some embodiments, A² is CHR⁶. In some embodiments, A² is NR⁷.

In some embodiments, W² is null. In some embodiments, W² is C₁₋₈ alkylene. In some embodiments, W² is C₁₋₄ alkylene. In some embodiments, W² is C₁₋₈ heteroalkylene. In some embodiments, W² is C₁₋₄ heteroalkylene.

In some embodiments, V² is null. In some embodiments, V² is C₁₋₈ alkylene. In some embodiments, V² is C₁₋₄ alkylene. In some embodiments, V² is C₁₋₈ heteroalkylene. In some embodiments, V² is C₁₋₄ heteroalkylene.

In some embodiments, A³ is null. In some embodiments, A³ is CHR⁸. In some embodiments, A³ is NR⁹.

In some embodiments, A³ and V² are null.

As generally described herein, R⁴ is H, C₁₋₈ alkyl, or C₁₋₈ heteroalkyl; R⁶ is H, C₁₋₈ alkyl, or C₁₋₈ heteroalkyl; R⁷ is H, C₁₋₈ alkyl, or C₁₋₈ heteroalkyl; R⁸ is H, C₁₋₈ alkyl, or C₁₋₈ heteroalkyl; R⁹ is H, C₁₋₈ alkyl, or C₁₋₈ heteroalkyl; and R¹⁰ is C₁₋₈ alkyl or C₁₋₈ heteroalkyl; wherein any two or more of R⁴, R⁶, R⁷, R⁸, and R⁹ can together form a ring.

In some embodiments, R⁴ is H. In some embodiments, R⁴ is C₁₋₈ alkyl. In some embodiments, R⁴ is C₁₋₄ alkyl. In some embodiments, R⁴ is C₁₋₈ heteroalkyl. In some embodiments, R⁴ is C₁₋₄ heteroalkyl.

In some embodiments, R⁶ is H. In some embodiments, R⁶ is C₁₋₈ alkyl. In some embodiments, R⁶ is C₁₋₄ alkyl. In some embodiments, R⁶ is C₁₋₈ heteroalkyl. In some embodiments, R⁶ is C₁₋₄ heteroalkyl.

In some embodiments, R⁷ is H. In some embodiments, R⁷ is C₁₋₈ alkyl. In some embodiments, R⁷ is C₁₋₄ alkyl. In some embodiments, R⁷ is C₁₋₈ heteroalkyl. In some embodiments, R⁷ is C₁₋₄ heteroalkyl.

In some embodiments, R⁸ is H. In some embodiments, R⁸ is C₁₋₈ alkyl. In some embodiments, R⁸ is C₁₋₄ alkyl. In some embodiments, R⁸ is C₁₋₈ heteroalkyl. In some embodiments, R⁸ is C₁₋₄ heteroalkyl.

In some embodiments, R⁹ is H. In some embodiments, R⁹ is C₁₋₈ alkyl. In some embodiments, R⁹ is C₁₋₄ alkyl. In some embodiments, R⁹ is C₁₋₈ heteroalkyl. In some embodiments, R⁹ is C₁₋₄ heteroalkyl.

In some embodiments, R¹⁰ is C₁₋₈ alkyl. In some embodiments, R¹⁰ is C₁₋₄ alkyl. In some embodiments, R¹⁰ is C₁₋₈ heteroalkyl. In some embodiments, R¹⁰ is C₁₋₄ heteroalkyl.

In some embodiments, R¹⁰ is

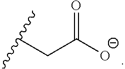 [SMILES: *CC(=O)[O-]]

In some embodiments, any two or more of R⁴, R⁶, R⁷, R⁸, and R⁹ together form a ring.

In some embodiments, R⁴ and R⁶ together form a ring. In some embodiments, R⁴ and R⁷ together form a ring. In some embodiments, R⁴ and R⁸ together form a ring. In some embodiments, R⁴ and R⁹ together form a ring. In some embodiments, R⁶ and R⁸ together form a ring. In some embodiments, R⁷ and R⁸ together form a ring. In some embodiments, R⁶ and R⁹ together form a ring.

In some embodiments, R⁷ and R⁹ together form a ring system. In some embodiments, R⁴, R⁶, and R⁸ together form a ring system.

In some embodiments, R⁴ and R⁶ together form a C₁₋₄ alkylene group. In some embodiments, R⁴ and R⁷ together form a C₁₋₄ alkylene group. In some embodiments, R⁴ and R⁸ together form a C₁₋₄ alkylene group. In some embodiments, R⁴ and R⁹ together form a C₁₋₄ alkylene group. In some embodiments, R⁶ and R⁸ together form a C₁₋₄ alkylene group. In some embodiments, R⁷ and R⁸ together form a C₁₋₄ alkylene group. In some embodiments, R⁶ and R⁹ together form a C₁₋₄ alkylene group. In some embodiments, R⁷ and R⁹ together form a C₁₋₄ alkylene group.

In some embodiments, R⁴, R⁶, and R⁸ together form a ring system having from 1-8 carbon atoms. In some embodiments, R⁴, R⁶, and R⁸ together form a ring system having from 4-8 carbon atoms.

In some embodiments, Ψ is covalently bound to the compound. In some embodiments, Ψ is selected from COOH, SO₃H, and PO₃H₂. In some embodiments, Ψ is selected from COO—, SO₃, and PO₃H—.

In some embodiments, Ψ is COOH. In some embodiments, Ψ is COO—.

In some embodiments, Ψ is not covalently bond to the compound. In some embodiments, Ψ is selected from halide, carboxylate, sulfate, nitrate, and phosphate.

In some embodiments, Ψ is halide. In some embodiments, Ψ is chloride or bromide. In some embodiments, Ψ is bromide.

### Formula (II-g)

In some embodiments, X is of Formula (II-g):

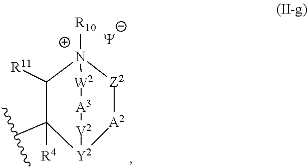

wherein:
   Y² is N, C₁₋₈ alkylene, or C₁₋₈ heteroalkylene;
   Z² is null, C₁₋₈ alkylene, or C₁₋₈ heteroalkylene;
   A² is null, CHR⁶ or NR⁷;
wherein A² and Z² cannot both be null;
   W² is null, C₁₋₈ alkylene, or C₁₋₈ heteroalkylene;
   V² is null, C₁₋₈ alkylene, or C₁₋₈ heteroalkylene;
   A³ is null, CHR⁸ or NR⁹;
wherein A², W², and V² cannot all be null;
   R⁴ is H, C₁₋₈ alkyl, or C₁₋₈ heteroalkyl;
   R⁶ is H, C₁₋₈ alkyl, or C₁₋₈ heteroalkyl;
   R⁷ is H, C₁₋₈ alkyl, or C₁₋₈ heteroalkyl;
   R⁸ is H, C₁₋₈ alkyl, or C₁₋₈ heteroalkyl;
   R⁹ is H, C₁₋₈ alkyl, or C₁₋₈ heteroalkyl;
   R¹⁰ is C₁₋₈ alkyl or C₁₋₈ heteroalkyl;
   R^{aa} is H, C₁₋₈ alkyl, or C₁₋₈ heteroalkyl; and
   Ψ represents an anionic group;
wherein any two or more of R⁴, R⁶, R⁷, R⁸, R⁹, and R¹¹ can together form a ring.

As generally described herein, Y² is N, C₁₋₈ alkylene, or C₁₋₈ heteroalkylene; Z² is null, C₁₋₈ alkylene, or C₁₋₈ heteroalkylene; A² is null, CHR⁶ or NR⁷; wherein A² and Z² cannot both be null; W² is null, C₁₋₈ alkylene, or C₁₋₈ heteroalkylene; V² is null, C₁₋₈ alkylene, or C₁₋₈ heteroalkylene; and A³ is null, CHR⁸ or NR⁹; wherein A², W², and V² cannot all be null.

In some embodiments, Y² is N. In some embodiments, Y² is C₁₋₈ alkylene. In some embodiments, Y² is C₁₋₄ alkylene. In some embodiments, Y² is C₁₋₈ heteroalkylene. In some embodiments, Y² is C₁₋₄ heteroalkylene.

In some embodiments, Z² is null. In some embodiments, Z² is C₁₋₈ alkylene. In some embodiments, Z² is C₁₋₄ alkylene. In some embodiments, Z² is C₁₋₈ heteroalkylene. In some embodiments, Z² is C₁₋₄ heteroalkylene.

In some embodiments, A² is null. In some embodiments, A² is CHR⁶. In some embodiments, A² is NR⁷.

In some embodiments, W² is null. In some embodiments, W² is C₁₋₈ alkylene. In some embodiments, W² is C₁₋₄ alkylene. In some embodiments, W² is C₁₋₈ heteroalkylene. In some embodiments, W² is C₁₋₄ heteroalkylene.

In some embodiments, V² is null. In some embodiments, V² is C₁₋₈ alkylene. In some embodiments, V² is C₁₋₄ alkylene. In some embodiments, V² is C₁₋₈ heteroalkylene. In some embodiments, V² is C₁₋₄ heteroalkylene.

In some embodiments, A³ is null. In some embodiments, A³ is CHR⁸. In some embodiments, A³ is NR⁹.

In some embodiments, A³ and V² are null.

As generally described herein, R⁴ is H, C₁₋₈ alkyl, or C₁₋₈ heteroalkyl; R⁶ is H, C₁₋₈ alkyl, or C₁₋₈ heteroalkyl; R⁷ is H, C₁₋₈ alkyl, or C₁₋₈ heteroalkyl; R⁸ is H, C₁₋₈ alkyl, or C₁₋₈ heteroalkyl; R⁹ is H, C₁₋₈ alkyl, or C₁₋₈ heteroalkyl; R¹⁰ is C₁₋₈ alkyl or C₁₋₈ heteroalkyl; and R¹¹ is H, C₁₋₈ alkyl, or C₁₋₈ heteroalkyl; wherein any two or more of R⁴, R⁶, R⁷, R⁸, R⁹, and R¹¹ can together form a ring.

In some embodiments, R⁴ is H. In some embodiments, R⁴ is C₁₋₈ alkyl. In some embodiments, R⁴ is C₁₋₄ alkyl. In some embodiments, R⁴ is C₁₋₈ heteroalkyl. In some embodiments, R⁴ is C₁₋₄ heteroalkyl.

In some embodiments, R⁶ is H. In some embodiments, R⁶ is C₁₋₈ alkyl. In some embodiments, R⁶ is C₁₋₄ alkyl. In some embodiments, R⁶ is C₁₋₈ heteroalkyl. In some embodiments, R⁶ is C₁₋₄ heteroalkyl.

In some embodiments, R⁷ is H. In some embodiments, R⁷ is C₁₋₈ alkyl. In some embodiments, R⁷ is C₁₋₄ alkyl. In some embodiments, R⁷ is C₁₋₈ heteroalkyl. In some embodiments, R⁷ is C₁₋₄ heteroalkyl.

In some embodiments, R⁸ is H. In some embodiments, R⁸ is C₁₋₈ alkyl. In some embodiments, R⁸ is C₁₋₄ alkyl. In some embodiments, R⁸ is C₁₋₈ heteroalkyl. In some embodiments, R⁸ is C₁₋₄ heteroalkyl.

In some embodiments, R⁹ is H. In some embodiments, R⁹ is C₁₋₈ alkyl. In some embodiments, R⁹ is C₁₋₄ alkyl. In some embodiments, R⁹ is C₁₋₈ heteroalkyl. In some embodiments, R⁹ is C₁₋₄ heteroalkyl.

In some embodiments, R¹⁰ is C₁₋₈ alkyl. In some embodiments, R¹⁰ is C₁₋₄ alkyl. In some embodiments, R¹⁰ is C₁₋₈ heteroalkyl. In some embodiments, R¹⁰ is C₁₋₄ heteroalkyl.

In some embodiments, R¹⁰ is

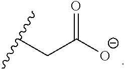 [SMILES: *CC(=O)[O-]]

In some embodiments, R¹¹ is H. In some embodiments, R¹¹ is C₁₋₈ alkyl. In some embodiments, R¹¹ is C₁₋₄ alkyl. In some embodiments, R¹¹ is C₁₋₈ heteroalkyl. In some embodiments, R¹¹ is C₁₋₄ heteroalkyl.

In some embodiments, any two or more of R⁴, R⁶, R⁷, R⁸, R⁹, and R¹¹ together form a ring.

In some embodiments, R⁴ and R⁶ together form a ring. In some embodiments, R⁴ and R⁷ together form a ring. In some embodiments, R⁴ and R⁸ together form a ring. In some embodiments, R⁴ and R⁹ together form a ring. In some embodiments, R⁴ and R⁸ together form a ring. In some embodiments, R⁴ and R¹¹ together form a ring. In some embodiments, R⁶ and R⁸ together form a ring. In some embodiments, R⁷ and R⁸ together form a ring. In some embodiments, R⁶ and R⁹ together form a ring.

In some embodiments, R⁷ and R⁹ together form a ring system. In some embodiments, R⁴, R⁶, and R⁸ together form a ring system.

In some embodiments, R⁴ and R⁶ together form a C₁₋₄ alkylene group. In some embodiments, R⁴ and R⁷ together form a C₁₋₄ alkylene group. In some embodiments, R⁴ and R⁸ together form a C₁₋₄ alkylene group. In some embodiments, R⁴ and R⁹ together form a C₁₋₄ alkylene group. In some embodiments, R⁴ and R¹¹ together form a C₁₋₄ alkylene group. In some embodiments, R⁶ and R⁸ together form a C₁₋₄ alkylene group. In some embodiments, R⁷ and R⁸ together form a C₁₋₄ alkylene group. In some embodiments, R⁶ and R⁹ together form a C₁₋₄ alkylene group. In some embodiments, R⁷ and R⁹ together form a C₁₋₄ alkylene group.

In some embodiments, R⁴, R⁶, and R⁸ together form a ring system having from 1-8 carbon atoms. In some embodiments, R⁴, R⁶, and R⁸ together form a ring system having from 4-8 carbon atoms.

In some embodiments, Ψ is covalently bound to the compound. In some embodiments, Ψ is selected from COOH, SO₃H, and PO₃H₂. In some embodiments, Ψ is selected from COO—, SO₃, and PO₃H—.

In some embodiments, Ψ is COOH. In some embodiments, Ψ is COO—.

In some embodiments, Ψ is not covalently bond to the compound. In some embodiments, Ψ is selected from halide, carboxylate, sulfate, nitrate, and phosphate.

In some embodiments, Ψ is halide. In some embodiments, Ψ is chloride or bromide.

In some embodiments, Ψ is bromide.

### ADDITIONAL EMBODIMENTS

In some embodiments, X has the formula:

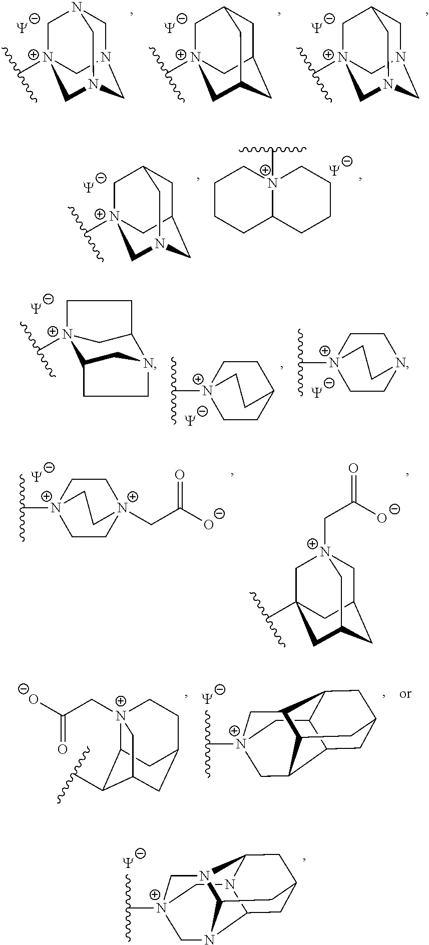

wherein Ψ represents an anionic group.

As generally described herein, Ψ represents an anionic group.

In some embodiments, Ψ is COOH. In some embodiments, Ψ is COO—.

In some embodiments, Ψ is halide. In some embodiments, Ψ is chloride or bromide.

In some embodiments, Ψ is bromide.

In some embodiments, X has the formula:

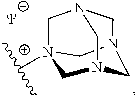

wherein Ψ represents an anionic group. In some embodiments, X has the formula:

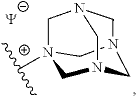

wherein Ψ represents halide. In some embodiments, X has the formula:

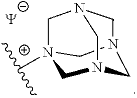

### L

In some embodiments, L comprises a C₁₋₁₂ alkylene, C₁₋₁₂ heteroalkylene, arylene, C₃₋₁₂ cycloalkylene, C₁₋₁₂ heterocyclylene, C₁₋₁₂ heteroarylene, polyethylene glycol, or a combination thereof.

In some embodiments, L comprises a C₁₋₁₂ alkylene group.

In some embodiments, L comprises a C₁₋₁₂ alkylene group comprising at least one double or triple bond.

In some embodiments, L has the formula —(CH₂)ₙ—, wherein n is 1, 2, 3, 4, 5, 6, 7, 8, 9, or 10. In some embodiments, n is 2, 3, 4, 5, 6, 7, or 8. In some embodiments, n is 2, 3, 4, 5, or 6. In some embodiments, n is 3, 4, or 5. In some embodiments, n is 5.

In some embodiments, L has the formula —(CH₂)ₙ—, wherein n is 1, 2, 3, 4, 5, 6, 7, 8, 9, or 10, preferably 2, 3, 4, 5, 6, 7, or 8, more preferably 2, 3, 4, 5, or 6, and especially preferably 3, 4, or 5.

In some embodiments, L comprises a butylene group, optionally substituted one or more times by —OH, ═O, or —COOH.

In some embodiments, L has the formula —CH₂CH₂CH₂CH₂—.

### Het

In some embodiments, Het comprises one or more heterocyclyl groups selected from pyrrolyl, furanyl, thiophenyl, imidazolyl, pyrazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, triazinyl and tetrazinyl, azepinyl, oxepinyl, thiepinyl, indolyl, isoindolyl, isatinyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, purinyl, naphthyridinyl, pteridinyl, quinolinyl, isoquinolinyl, cinnolinyl, quinoxalinyl, phthalazinyl, quinazolinyl, phenanthridinyl, dibenzofuranyl, carbazolyl, acridinyl, phenothiazinyl, phenoxazinyl, alloxazinyl, dibenzazepinyl, dibenzoxazepinyl, thioxanthenyl, and phenazinyl.

In some embodiments, Het is of Formula (III):

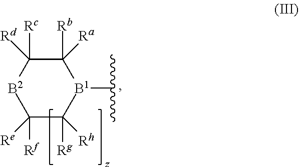

wherein:
z is 1 or 0;
B¹ is N, CRⁱ, C₂₋₃ alkylene, or C₁₋₃ heteroalkylene;
B² is null, O, S, NR^{j}, C(R^{k}R^{k}*), C(R^{k}R^{k}*)C(R^{l}R^{l}*), C(R^{k}R^{k}*)C(R^{l}R^{l}*)C(R^{m}R^{m}*) or C₁₋₃ heteroalkylene;
each of R^{a}, R^{b}, R^{c}, R^{d}, R^{e}, R^{f}, R^{g}, R^{h}, Rⁱ, R^{j}, R^{k}, R^{k}*, R^{l}, R^{l}*, R^{m}, and R^{m}* is independently selected from H, F, Cl, Br, I, OH, COOH, C(═O)O(C₁₋₆ alkyl), NH₂, N(C₁₋₆ alkyl)₂, NH(C₁₋₆ alkyl), C₁₋₆ alkyl, C₁₋₆ heteroalkyl, aryl, C₃₋₁₂ cycloalkyl, C₁₋₁₂ heterocyclyl, or C₁₋₁₂ heteroaryl, wherein said C₁₋₆ alkyl, C₁₋₁₂ heteroalkyl, aryl, C₃₋₁₂ cycloalkyl, C₁₋₁₂ heterocyclyl, and C₁₋₁₂ heteroaryl can be substituted one or more times by F, Cl, Br, I, OH, COOH, C(═O)O)C₁₋₄ alkyl), NH₂, N(C₁₋₄ alkyl)₂, NH(C₁₋₄ alkyl), C₁₋₄ alkyl, or C₁₋₄ heteroalkyl;
any two or more of R^{a}, R^{b}, R^{c}, R^{d}, R^{e}, R^{f}, R^{g}, R^{h}, Rⁱ, R^{j}, B¹, and B² can together form a ring;
any two geminal R groups can together form an oxo, a thiooxo, an imino, or an exo-olefin; and
any two R groups on adjacent carbon atoms can together form a double bond.

As generally described herein, z is 0 or 1. In some embodiments, z is 0. In some embodiments, z is 1.

In some embodiments of Formula (III), the Het is of Formula (III-a) and z is 0:

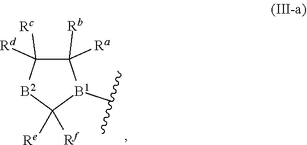

### or the Het is of Formula (III-b) and z is 1:

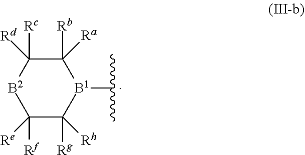

In some embodiments of Formula (III), R^{a}, R^{b}, R^{c}, and R^{d} together form an aromatic or heteroaromatic ring of Formula (IV):

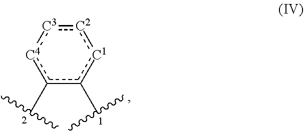

wherein
C¹ is null, O, S, N, CR^{A}r, or N(RAr¹*)_{y};
C² is null, O, S, N, CR^{Ar2}, or N(RAr²*)_{y};
C³ is null, O, S, N, CR^{Ar3}, or N(RAr³*)_{y};
C⁴ is null, O, S, N, CR^{Ar4}, or N(RAr⁴*)_{y};
y is in each case selected from 0 or 1;
[symbol] represents a single or double bond;
wavy line 1 indicates the bond to B¹;
wavy line 2 indicates the bond to B²;
each of R^{Ar1}, R^{Ar2}, R^{Ar3}, and R^{Ar4} is independently selected from H, F, Cl, Br, I, COOH, OH, NH₂, C₁₋₄ alkyl, C₁₋₄ heteroalkyl, O(C₁₋₄ alkyl), O(C₁₋₄ heteroalkyl), NH(C₁₋₄ alkyl), NH(C₁₋₄ heteroalkyl), N(C₁₋₄ alkyl)(C₁₋₄ heteroalkyl), N(C₁₋₄ alkyl)₂, N(C₁₋₄ heteroalkyl)₂; and
each of R^{Ar1}*, R^{Ar2}*, R^{Ar3}*, and R^{Ar4}* is independently selected from H, C₁₋₄ alkyl, and C₁₋₄ heteroalkyl;
wherein any two or more of R^{Ar1}, R^{Ar2}, R^{Ar3}, and R^{Ar4} can together form a ring, and wherein any of R^{Ar1}, R^{Ar1}*, R^{Ar4}, or R^{Ar4}* can form a ring with any one or more of R^{e}, R^{f}, R^{g}, R^{h}, B¹, and B².

In some embodiments of Formula (IV), R^{a}, R^{b}, R^{c}, and R^{d} together form a fused benzo ring of Formula (IV-a):

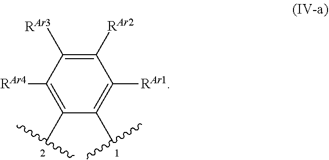

In some embodiments of Formula (III), R^{e}, R^{f}, R⁹, and R^{h} together form an aromatic or heteroaromatic ring of Formula (V):

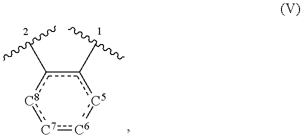

wherein
C⁵ is null, O, S, N, CR^{Ar5}, or N(RAr⁵*)_{y};
C⁶ is null, O, S, N, CR^{Ar6}, or N(RAr⁶*)_{y};
C⁷ is null, O, S, N, CR^{Ar7}, or N(RAr⁷*)_{y};
C⁸ is null, O, S, N, CR^{Ar8}, or N(RAr⁸*)_{y};
y is in each case selected from 0 or 1;
[symbol] represents a single or double bond;
wavy line 1 indicates the bond to B¹;
wavy line 2 indicates the bond to B²;
each of R^{Ar5}, R^{Ar6}, R^{Ar7}, and R^{Ar8} is independently selected from H, F, Cl, Br, I, COOH, OH, NH₂, C₁₋₄ alkyl, C₁₋₄ heteroalkyl, O(C₁₋₄ alkyl), O(C₁₋₄ heteroalkyl), NH(C₁₋₄ alkyl), NH(C₁₋₄ heteroalkyl), N(C₁₋₄ alkyl)(C₁₋₄ heteroalkyl), N(C₁₋₄ alkyl)₂, N(C₁₋₄ heteroalkyl)₂; and
each of R^{Ar5}*, R^{Ar6}*, R^{Ar7}*, and R^{Ar8}* is independently selected from H, C₁₋₄ alkyl, and C₁₋₄ heteroalkyl;
wherein any two or more of R^{Ar5}, R^{Ar6}, R^{Ar7}, and R^{Ar8} can together form a ring, and wherein any of R^{Ar5}, R^{Ar5}*, R^{Ar8}, or R^{Ar8}* can form a ring with any one or more of R^{a}, R^{b}, R^{c}, R^{d}, B1, and B².

In some embodiments of Formula (V), R^{e}, R^{f}, R⁹, and R^{h} together form a fused benzo ring of Formula (V-a):

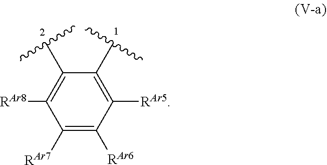

In some embodiments of Formula (III-a) wherein R^{a}, R^{b}, R^{c}, and R^{d} together form a fused benzo ring of Formula (IV-a), Het is of Formula (III-a-1):

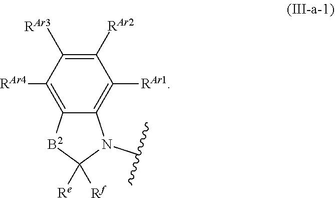

In some embodiments of Formula (III-b) wherein R^{a}, R^{b}, R^{c}, and R^{d} together form a fused benzo ring of Formula (IV-a) and R^{e}, R^{f}, R⁹, and R^{h} together form a fused benzo ring of Formula (V-a), Het is of Formula (III-b-1):

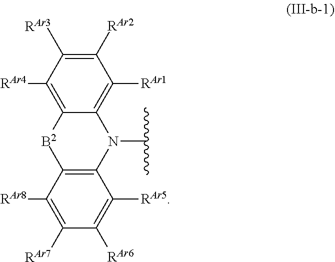

As generally described herein, B¹ is N, CRⁱ, C₂₋₃ alkylene, or C₁₋₃ heteroalkylene; B² is null, O, S, NR^{j}, C(R^{k}R^{k}*), C(R^{k}R^{k}*)C(R^{l}R^{l}*), C(R^{k}R^{k}*)C(R^{l}R^{l}*)C(R^{m}R^{m}*) or C₁₋₃ heteroalkylene; each of R^{a}, R^{b}, R^{c}, R^{d}, R^{e}, R^{f}, R^{g}, R^{h}, Rⁱ, R^{j}, R^{k}, R^{k}*, R^{l}, R^{l}*, R^{m}, and R^{m}* is independently selected from H, F, Cl, Br, I, OH, COOH, C(═O)O(C₁₋₆ alkyl), NH₂, N(C₁₋₆ alkyl)₂, NH(C₁₋₆ alkyl), C₁₋₆ alkyl, C₁₋₆ heteroalkyl, aryl, C₃₋₁₂ cycloalkyl, C₁₋₁₂ heterocyclyl, or C₁₋₁₂ heteroaryl, wherein said C₁₋₆ alkyl, C₁₋₁₂ heteroalkyl, aryl, C₃₋₁₂ cycloalkyl, C₁₋₁₂ heterocyclyl, and C₁₋₁₂ heteroaryl can be substituted one or more times by F, Cl, Br, I, OH, COOH, C(═O)O)C₁₋₄ alkyl), NH₂, N(C₁₋₄ alkyl)₂, NH(C₁₋₄ alkyl), C₁₋₄ alkyl, or C₁₋₄ heteroalkyl; any two or more of R^{a}, R^{b}, R^{c}, R^{d}, R^{e}, R^{f}, R^{g}, R^{h}, Rⁱ, R^{j}, B¹, and B² can together form a ring; any two geminal R groups can together form an oxo, a thiooxo, an imino, or an exo-olefin; and any two R groups on adjacent carbon atoms can together form a double bond.

In some embodiments, B¹ is N or CR¹. In some embodiments, B¹ is N.

In some embodiments, B² is null. In some embodiments, B² is NR^{j}. In some embodiments, B² is S. In some embodiments, B² is O. In some embodiments, B² is NR^{j}; and R^{j} and R^{e} together form a double bond.

In some embodiments, R^{e} and R^{f} together form an oxo.

In some embodiments, B¹ is CRⁱ; Rⁱ and R^{e} together form a double bond; and B² is S, O, or NR^{j}.

In some embodiments, B¹ is N; and B² is S, O, or NR^{j}. In some embodiments, B¹ is N; and B² is S.

In some embodiments, R^{e} and R^{f} together form an oxo; B² is C(R^{k}R^{k}*); and R^{k} and R^{k}* together form an oxo. In some embodiments, B¹ is N; R^{e} and R^{f} together form an oxo; B² is C(R^{k}R^{k}*); and R^{k} and R^{k}* together form an oxo.

As generally described herein, C¹ is null, O, S, N, CR^{Ar1}, or N(RAr¹*)_{y}; C² is null, O, S, N, CR^{Ar2}, or N(RAr²*)_{y}; C³ is null, O, S, N, CR^{Ar3}, or N(RAr³*)_{y}; C⁴ is null, O, S, N, CR^{Ar4} or N(RAr⁴*)_{y}; and y is in each case selected from 0 or 1.

As generally described herein, each of R^{Ar1}, R^{Ar2}, R^{Ar3}, and R^{Ar4} is independently selected from H, F, Cl, Br, I, COOH, OH, NH₂, C₁₋₄ alkyl, C₁₋₄ heteroalkyl, O(C₁₋₄ alkyl), O(C₁₋₄ heteroalkyl), NH(C₁₋₄ alkyl), NH(C₁₋₄ heteroalkyl), N(C₁₋₄ alkyl)(C₁₋₄ heteroalkyl), N(C₁₋₄ alkyl)₂, and N(C₁₋₄ heteroalkyl)₂; and each of R^{Ar1}*, R^{Ar2}*, R^{Ar3}*, and R^{Ar4}* is independently selected from H, C₁₋₄ alkyl, and C₁₋₄ heteroalkyl; wherein any two or more of R^{Ar1}, R^{Ar2}, R^{Ar3}, and R^{Ar4} can together form a ring, and wherein any of R^{Ar1}, R^{Ar1}*, R^{Ar4}, or R^{Ar4}* can form a ring with any one or more of R^{e}, R^{f}, R^{g}, R^{h}, B¹, and B².

In some embodiments, C¹ is CR^{Ar1}; C² is CR^{Ar2}; C³ is CR^{Ar3}; and C⁴ is CR^{Ar4}.

In some embodiments, R^{Ar1} is H or Cl. In some embodiments, R^{Ar1} is H. In some embodiments, R^{Ar1} is Cl. In some embodiments, R^{Ar2} is H. In some embodiments, R^{Ar3} is H.

In some embodiments, R^{Ar4} is H. In some embodiments, R^{Ar1} is H, R^{Ar2} is H, R^{Ar3} is H, and RAr⁴ is H. In some embodiments, R^{Ar1} is Cl, RAr² is H, RAr³ is H, and RAr⁴ is H.

As generally described herein, C⁵ is null, O, S, N, CR^{Ar5}, or N(RAr⁵*)_{y}; C⁶ is null, O, S, N, CR^{Ar6}, or N(RAr⁶*)_{y}; C⁷ is null, O, S, N, CR^{Ar7}, or N(RAr⁷*)_{y}; C⁸ is null, O, S, N, CR^{Ar8}, or N(RAr⁸*)_{y}; and y is in each case selected from 0 or 1.

As generally described herein, each of R^{Ar5}, R^{Ar6}, R^{Ar7}, and R^{Ar8} is independently selected from H, F, Cl, Br, I, COOH, OH, NH₂, C₁₋₄ alkyl, C₁₋₄ heteroalkyl, O(C₁₋₄ alkyl), O(C₁₋₄ heteroalkyl), NH(C₁₋₄ alkyl), NH(C₁₋₄ heteroalkyl), N(C₁₋₄ alkyl)(C₁₋₄ heteroalkyl), N(C₁₋₄ alkyl)₂, and N(C₁₋₄ heteroalkyl)₂; and each of R^{Ar5}*, R^{Ar6}*, R^{Ar7}*, and R^{Ar8}* is independently selected from H, C₁₋₄ alkyl, and C₁₋₄ heteroalkyl; wherein any two or more of R^{Ar5}, R^{Ar6}, R^{Ar7}, and R^{Ar8} can together form a ring, and wherein any of R^{Ar5}, R^{Ar5}*, R^{Ar8}, or R^{Ar8}* can form a ring with any one or more of R^{a}, R^{b}, R^{c}, R^{d}, B¹, and B².

In some embodiments, C⁵ is CR^{Ar5}; C⁶ is CR^{Ar6}; C⁷ is CR^{Ar7}; and C⁸ is CR^{Ar8}.

In some embodiments, RAr⁵ is H or Cl. In some embodiments, R^{Ar5} is H. In some embodiments, R^{Ar5} is Cl. In some embodiments, R^{Ar6} is H. In some embodiments, R^{Ar7} is H.

In some embodiments, R^{Ar8} is H. In some embodiments, R^{Ar5} is H, R^{Ar6} is H, R^{Ar7} is H, and R^{Ar8} is H. In some embodiments, RAr⁵ is Cl, R^{Ar6} is H, R^{Ar7} is H, and R^{Ar8} is H.

In some embodiments, R^{Ar1} is H, R^{Ar2} is H, R^{Ar3} is H, R^{Ar4} is H, R^{Ar5} is Cl, RAr⁶ is H, R^{Ar7} is H, and RAr⁸ is H.

In some embodiments, B² is S, R^{Ar1} is H, R^{Ar2} is H, R^{Ar3} is H, R^{Ar4} is H, R^{Ar5} is Cl, R^{Ar6} is H, R^{Ar7} is H, and R^{Ar8} is H.

In some embodiments, B¹ is N, R^{Ar1} is H, R^{Ar2} is H, R^{Ar3} is H, R^{Ar4} is H, R^{Ar5} is Cl, R^{Ar6} is H, R^{Ar7} is H, and RAr⁸ is H.

In some embodiments, B¹ is N, B² is S, R^{Ar1} is H, R^{Ar2} is H, R^{Ar3} is H, R^{Ar4} is H, R^{Ar5} is Cl, R^{Ar6} is H, R^{Ar7} is H, and R^{Ar8} is H.

In some embodiments, Het is

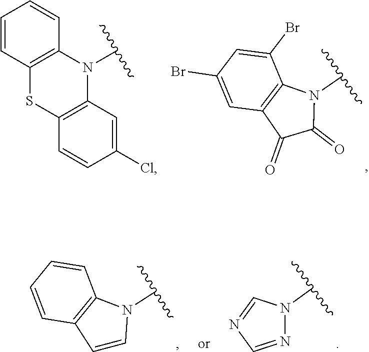 [SMILES: *N1c2ccccc2Sc2ccc(Cl)cc21 ; *N1C(=O)C(=O)c2cc(Br)cc(Br)c21 ; *n1ccc2ccccc21 ; *n1cncn1]

In some embodiments, Het is

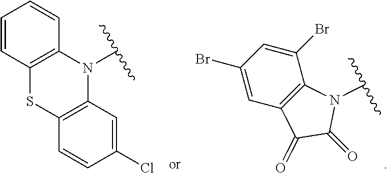 [SMILES: *N1c2ccccc2Sc2ccc(Cl)cc21 ; *N1C(=O)C(=O)c2cc(Br)cc(Br)c21]

In some embodiments, Het is

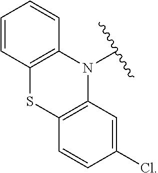 [SMILES: *N1c2ccccc2Sc2ccc(Cl)cc21]

### Additional Embodiments

In some embodiments, the compound of Formula (I) is of Formula (I-a):

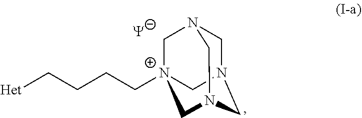

or a pharmaceutically acceptable salt thereof.

In some embodiments, the compound of Formula (I) is of Formula (I-b):

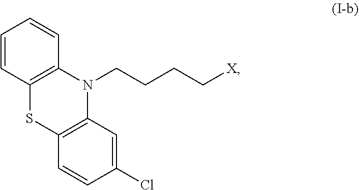

or a pharmaceutically acceptable salt thereof.

In some embodiments, the compound of Formula (I) is of formula:

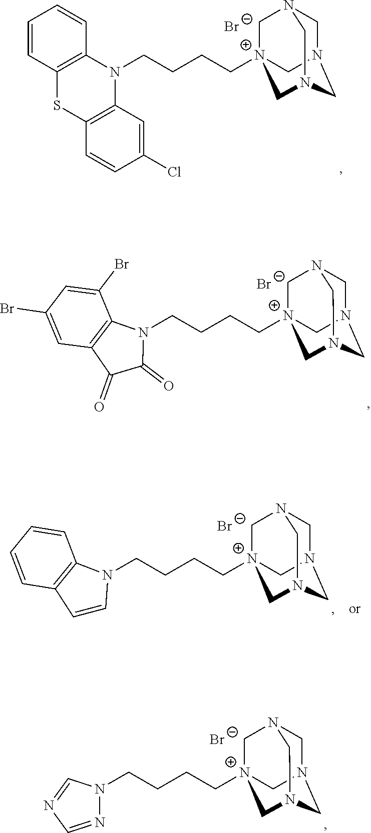

or a pharmaceutically acceptable salt thereof.

In some embodiments, the present disclosure provides compounds in the free-base form of the compounds of any of the formulae herein (e.g., free-base form of Formula (I)).

### Pharmaceutical Compositions

In another aspect, the present disclosure provides a pharmaceutical composition, comprising a compound of any of the formulae herein (e.g., Formula (I)), or a pharmaceutically acceptable salt thereof, and a pharmaceutically acceptable excipient. In certain embodiments, a compound described herein is provided in an effective amount in the pharmaceutical composition.

In some embodiments, the pharmaceutical composition comprises a second therapeutic agent. In some embodiments, the second therapeutic agent is a cancer chemotherapeutic.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Relative amounts of the active ingredient, the pharmaceutically acceptable carrier or excipient, and/or any additional ingredients in a pharmaceutical composition described herein will vary, depending upon the identity, size, and/or condition of the subject treated and further depending upon the route by which the composition is to be administered.

Pharmaceutically acceptable carriers/excipients used in the manufacture of provided pharmaceutical compositions include inert diluents, solvents, dispersing and/or granulating agents, surface active agents and/or emulsifiers, disintegrating agents, binding agents, preservatives, buffering agents, lubricating agents, oils, butters, and/or waxes. Excipients such as coloring agents, coating agents, sweetening agents, flavoring agents, and fragrances may also be present in the composition.

The compounds and compositions provided herein can be administered by any route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein.

A compound or composition, as described herein, can be administered in combination with one or more additional pharmaceutical agents. The compounds or compositions can be administered in combination with additional pharmaceutical agents that improve their activity in treating a disease in a subject in need thereof, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects.

### Methods of Activating Protein Phosphatase 2A and Treating Cancer

In another aspect, the present disclosure provides a method of activating protein phosphatase 2A, comprising administering to a patient in need thereof a compound of any of the formula herein (e.g., Formula (I)), or a pharmaceutically acceptable salt thereof. In some embodiments, the method comprises agonism of protein phosphatase 2A. In some embodiments, the method comprises activation of protein phosphatase 2A.

In another aspect, the present disclosure provides a method of treating cancer, comprising administering to a patient in need thereof a compound of any of the formula herein (e.g., Formula (I)), or a pharmaceutically acceptable salt thereof.

In some embodiments, the cancer is acute lymphoblastic leukemia (ALL), acute myeloid leukemia (AML), cancer in adrenocortical carcinoma, adrenal cortex cancer, AIDS-related cancers, Kaposi sarcoma, AIDS-related lymphoma, primary CNS lymphoma, anal cancer, appendix cancer, carcinoid tumors, astrocytomas, atypical teratoid/rhabdoid tumor, basal cell carcinoma, skin cancer (nonmelanoma), bile duct cancer, extrahepatic bladder cancer, bladder cancer, bone cancer (includes Ewing sarcoma and osteosarcoma and malignant fibrous histiocytoma), brain tumors, breast cancer, bronchial tumors, Burkitt lymphoma (non-Hodgkin), carcinoid tumor, cardiac (heart) tumors, atypical teratoid/rhabdoid tumor, embryonal tumors, germ cell tumors, lymphoma, primary—cervical cancer, cholangiocarcinoma, chordoma, chronic lymphocytic leukemia (CLL), chronic myelogenous leukemia (CML), chronic myeloproliferative neoplasms, colorectal cancer, colorectal cancer, craniopharyngioma, cutaneous T-cell lymphoma, ductal carcinoma in situ (DCIS), embryonal tumors, central nervous system, endometrial cancer, ependymoma, esophageal, esthesioneuroblastoma, ewing sarcoma, extracranial germ cell tumor, extragonadal germ cell tumor, eye cancer, intraocular melanoma, retinoblastoma, fallopian tube cancer, fibrous histiocytoma of bone, malignant, and osteosarcoma, gallbladder cancer, gastric (stomach) cancer, gastric (stomach) cancer, gastrointestinal carcinoid tumor, gastrointestinal stromal tumors (GIST), gastrointestinal stromal tumors (GIST), germ cell tumors, central nervous system, extracranial, extragonadal, ovarian testicular, gestational trophoblastic disease, gliomas, hairy cell leukemia, head and neck cancer, heart tumors, hepatocellular (liver) cancer, histiocytosis, Langerhans Cell, Hodgkin's lymphoma, hypopharyngeal cancer, intraocular melanoma, islet cell tumors, pancreatic neuroendocrine tumors, Kaposi sarcoma, kidney—langerhans cell histiocytosis, laryngeal cancer, laryngeal cancer and papillomatosis, leukemia, lip and oral cavity cancer, liver cancer (primary), lung cancer, lung cancer, lymphoma—macroglobulinemia, Waldenström-Non-Hodgkin lymphoma, male breast cancer, malignant fibrous histiocytoma of bone and osteosarcoma, melanoma, intraocular (eye), Merkel cell carcinoma, mesothelioma, malignant, mesothelioma, metastatic squamous neck cancer with occult primary, midline tract carcinoma involving NUT gene, mouth cancer, multiple endocrine neoplasia syndromes, multiple myeloma/plasma cell neoplasms, mycosis fungoides, myelodysplastic syndromes, myelodysplastic/myeloproliferative neoplasms and chronic myeloproliferative neoplasms, myelogenous leukemia, chronic (CML), myeloid leukemia, acute (AML), nasal cavity and paranasal sinus cancer, nasopharyngeal cancer, nasopharyngeal cancer, neuroblastoma, non-hodgkin lymphoma, non-small cell lung cancer, oral cancer, lip and oral cavity cancer and oropharyngeal cancer, osteosarcoma and malignant fibrous histiocytoma of bone, ovarian cancer, pancreatic cancer and pancreatic neuroendocrine tumors (islet cell tumors), papillomatosis, paraganglioma, paraganglioma, paranasal sinus and nasal cavity cancer, parathyroid cancer, penile cancer, pharyngeal cancer, pheochromocytoma, pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, renal cell (kidney) cancer, retinoblastoma, rhabdomyosarcoma, salivary gland cancer, salivary gland tumors, Ewing sarcoma, Kaposi sarcoma, osteosarcoma, rhabdomyosarcoma, uterine sarcoma, vascular tumors, Sézary syndrome, skin cancer, small cell lung cancer, small intestine cancer, soft tissue sarcoma, squamous cell carcinoma, squamous neck cancer with occult primary, metastatic, stomach (gastric) cancer, stomach (gastric) cancer, T-cell lymphoma, cutaneous, testicular cancer, throat cancer, thymoma and thymic carcinoma, thyroid cancer, ureter and renal pelvis, transitional cell cancer, urethral cancer, uterine cancer, endometrial and uterine sarcoma, vaginal cancer, vaginal cancer, vascular tumors, vulvar cancer, Waldenström Macroglobulinemia, or Wilms Tumor.

In some embodiments, the cancer is colorectal cancer. In some embodiments, the cancer is colorectal cancer that has been previously treated with 5-fluorouracil, folinic acid, oxaliplatin, or a combination thereof. In some embodiments, the cancer is colorectal cancer that has been previously treated with 5-fluorouracil, folinic acid, and oxaliplatin. In some embodiments, the cancer is colorectal cancer that is resistant to one or more of 5-fluorouracil, folinic acid, and oxaliplatin.

In some embodiments, the method further comprises administering to the patient at least one additional therapy. In some embodiments, the at least one additional therapy comprises surgery, radiation therapy, immunotherapy, chemotherapy, or a combination thereof.

In some embodiments, the method further comprises administering to the patient at least one additional therapeutic agent. In some embodiments, the at least one additional therapeutic agent comprises a cancer chemotherapeutic agent.

In some embodiments, the at least one additional therapeutic agent comprises a nucleoside analogue, antifolate, antimetabolite, topoisomerase I inhibitor, anthracycline, podophyllotoxin, taxanes, vinca alkaloid, alkylating agent, platinum compound, proteasome inhibitor, nitrogen mustard, estrogen analogue, monoclonal antibody, tyrosine kinase inhibitor, mTOR inhibitor, retinoid, immunomodulatory agent, histone deacetylase inhibitor, other kinase inhibitor, metabolic inhibitors, microtubule inhibitors, or combination thereof.

In some embodiments, the at least one additional therapeutic agent comprises abiraterone acetate, methotrexate, paclitaxel albumin-stabilized nanoparticle, brentuximab vedotin, ado-trastuzumab emtansine, doxorubicin hydrochloride, afatinib dimaleate, everolimus, netupitant, palonosetron hydrochloride, imiquimod, aldesleukin, alectinib, alemtuzumab, melphalan hydrochloride, melphalan, pemetrexed disodium, chlorambucil, aminolevulinic acid, anastrozole, aprepitant, pamidronate disodium, exemestane, nelarabine, arsenic trioxide, ofatumumab, asparaginase Erwinia chrysanthemi, atezolizumab, bevacizumab, axitinib, azacitidine, carmustine, belinostat, bendamustine hydrochloride, bevacizumab, bexarotene, tositumomab, bicalutamide, bleomycin, blinatumomab, blinatumomab, bortezomib, bosutinib, busulfan, cabazitaxel, cabozantinib, alemtuzumab, irinotecan hydrochloride, capecitabine, fluorouracil, carboplatin, carfilzomib, bicalutamide, lomustine, ceritinib, daunorubicin hydrochloride, cetuximab, chlorambucil, cyclophosphamide, clofarabine, cobimetinib, dactinomycin, cobimetinib, crizotinib, ifosfamide, ramucirumab, cytarabine, dabrafenib, dacarbazine, decitabine, daratumumab, dasatinib, daunorubicin hydrochloride, decitabine, efibrotide sodium, defibrotide sodium, degarelix, denileukin diftitox, denosumab, dexamethasone, dexrazoxane hydrochloride, dinutuximab, docetaxel, doxorubicin hydrochloride, dacarbazine, rasburicase, epirubicin hydrochloride, elotuzumab, oxaliplatin, eltrombopag olamine, aprepitant, elotuzumab, enzalutamide, epirubicin hydrochloride, cetuximab, eribulin mesylate, vismodegib, erlotinib hydrochloride, etoposide, raloxifene hydrochloride, melphalan hydrochloride, toremifene, panobinostat, fulvestrant, letrozole, filgrastim, fludarabine phosphate, flutamide, methotrexate, pralatrexate, obinutuzumab, gefitinib, gemcitabine, gemcitabine hydrochloride, gemtuzumab ozogamicin, afatinib dimaleate, imatinib mesylate, glucarpidase, goserelin acetate, eribulin mesylate, trastuzumab, topotecan hydrochloride, palbociclib, ibritumomab tiuxetan, ibrutinib, ponatinib hydrochloride, idarubicin hydrochloride, idelalisib, imiquimod, axitinib, recombinant interferon alfa-2b, tositumomab, ipilimumab, gefitinib, romidepsin, ixabepilone, ixazomib citrate, ruxolitinib phosphate, cabazitaxel, ado-trastuzumab emtansine, palifermin, pembrolizumab, lanreotide acetate, lapatinib ditosylate, lenalidomide lenvatinib mesylate, leuprolide acetate, olaparib, vincristine sulfate, procarbazine hydrochloride, mechlorethamine hydrochloride, megestrol acetate, trametinib, mercaptopurine, temozolomide, mitoxantrone hydrochloride, plerixafor, busulfan, azacitidine, gemtuzumab ozogamicin, vinorelbine tartrate, necitumumab, nelarabine, sorafenib tosylate, nilotinib, ixazomib citrate, nivolumab, romiplostim, obinutuzumab, ofatumumab, olaparib, omacetaxine mepesuccinate, pegaspargase, ondansetron hydrochloride, osimertinib, panitumumab, panobinostat, peginterferon alfa-2b, pembrolizumab, pertuzumab, plerixafor, pomalidomide, ponatinib hydrochloride, necitumumab, pralatrexate, procarbazine hydrochloride, aldesleukin, denosumab, ramucirumab, rasburicase, regorafenib, lenalidomide, rituximab, rolapitant hydrochloride, romidepsin, ruxolitinib phosphate, siltuximab, dasatinib, sunitinib malate, thalidomide, dabrafenib, osimertinib, talimogene, atezolizumab, temsirolimus, thalidomide, dexrazoxane hydrochloride, trabectedin, trametinib, trastuzumab, lapatinib ditosylate, dinutuximab, vandetanib, rolapitant hydrochloride, bortezomib, venetoclax, crizotinib, enzalutamide, ipilimumab, trabectedin, ziv-aflibercept, idelalisib, ceritinib, or combination thereof.

In some embodiments, the at least one additional therapeutic agent is gemcitabine or gemcitabine hydrochloride.

In some embodiments, the cancer chemotherapeutic agent is selected from 5-fluorouracil, capecitabine, irinotecan, oxaliplatin, trifluridine, tipiracil, and combinations thereof.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Chemical Synthesis

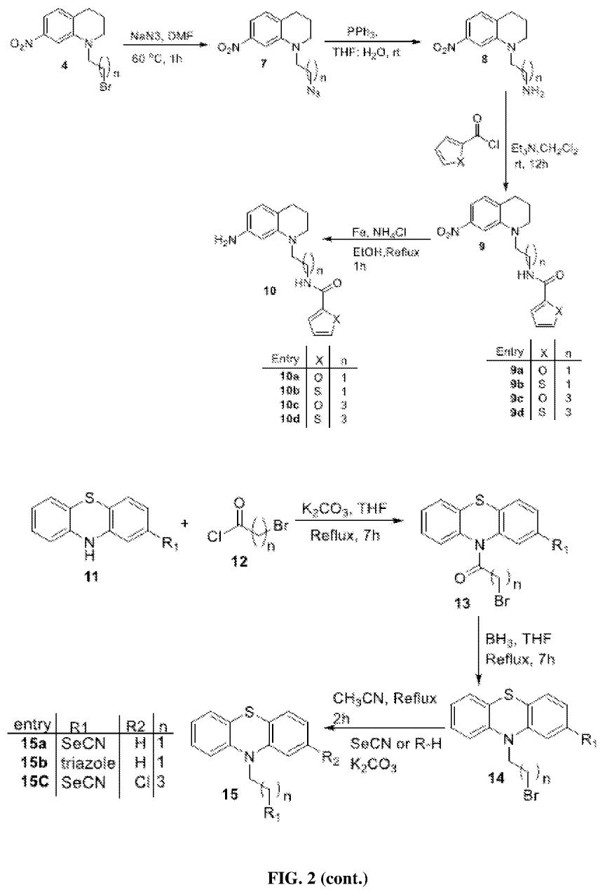

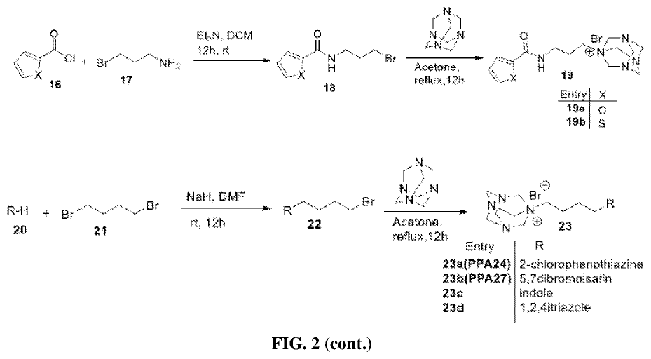

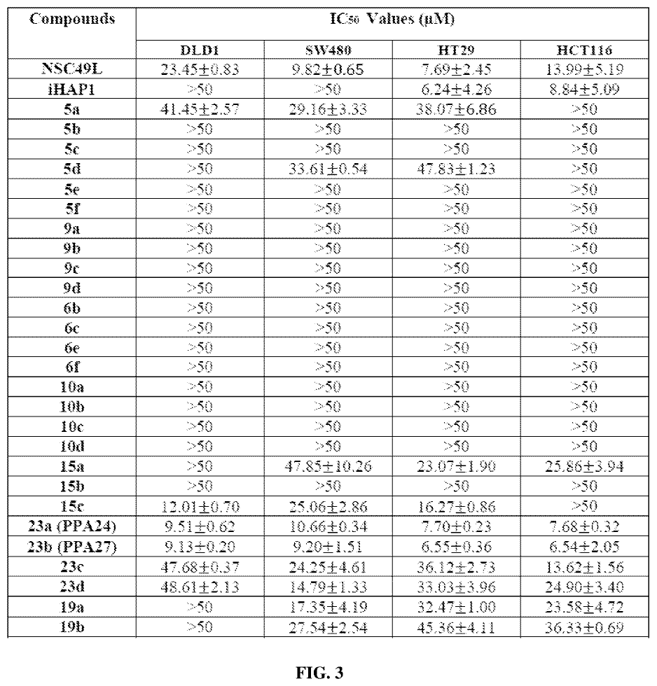

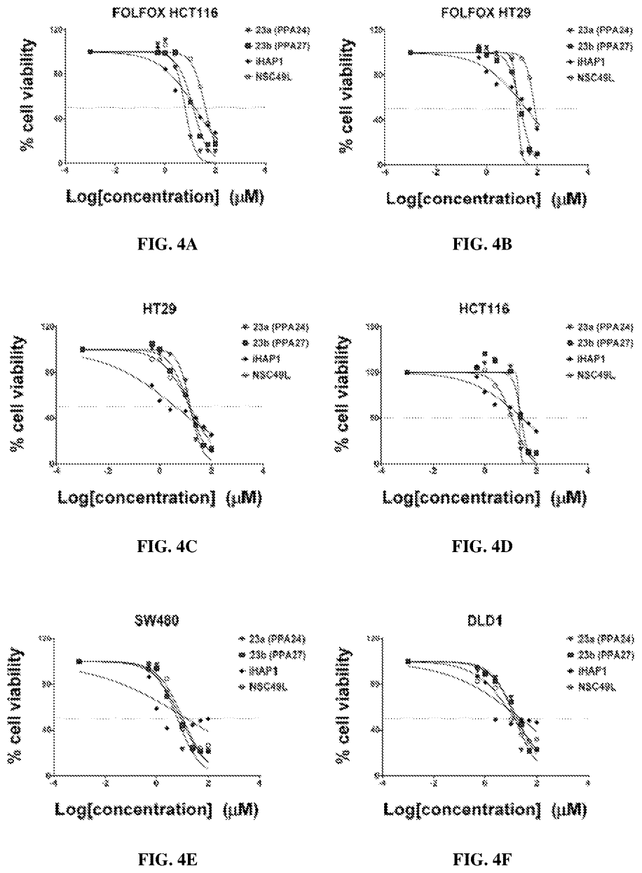

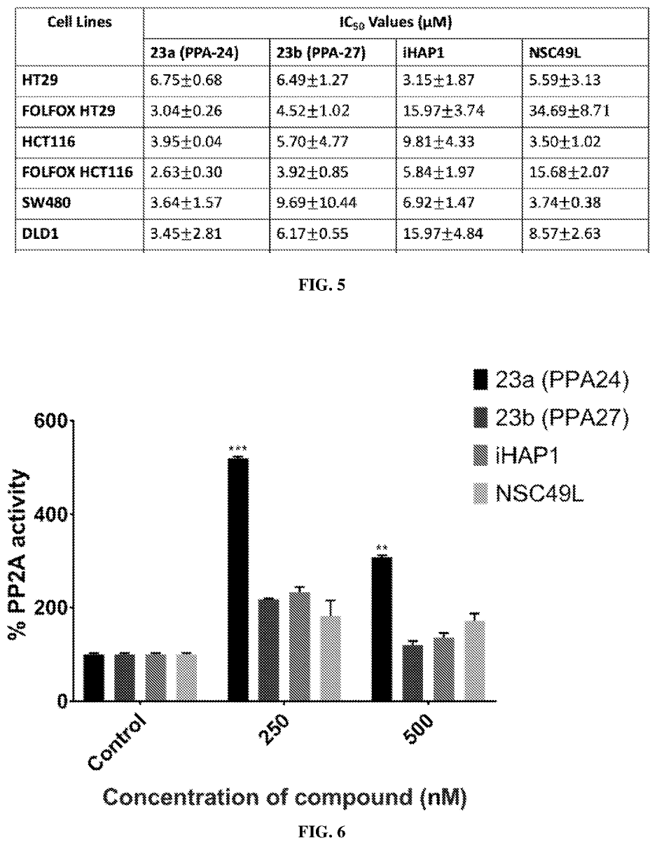

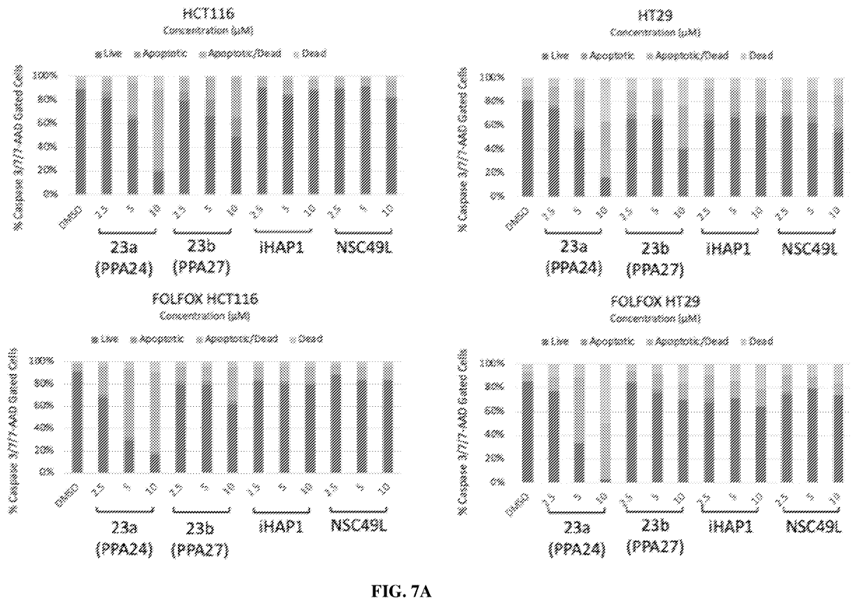

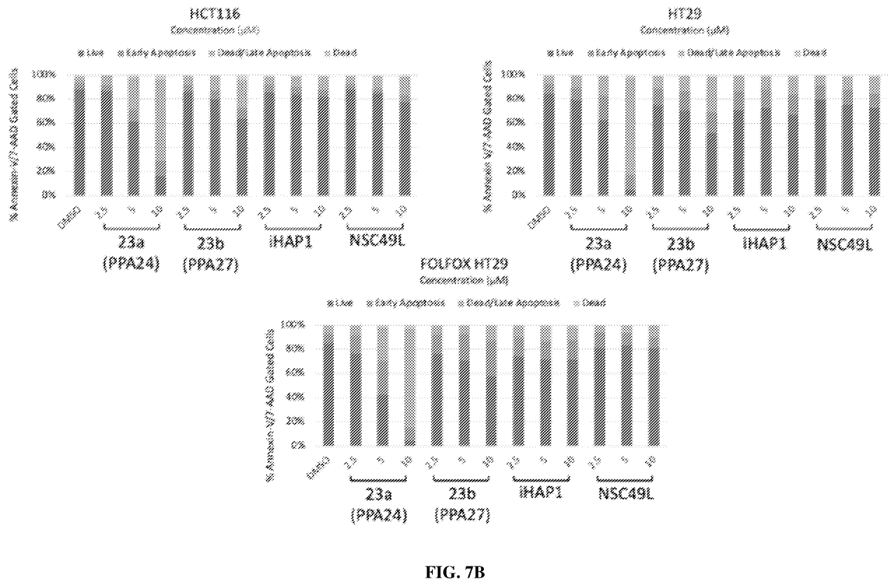

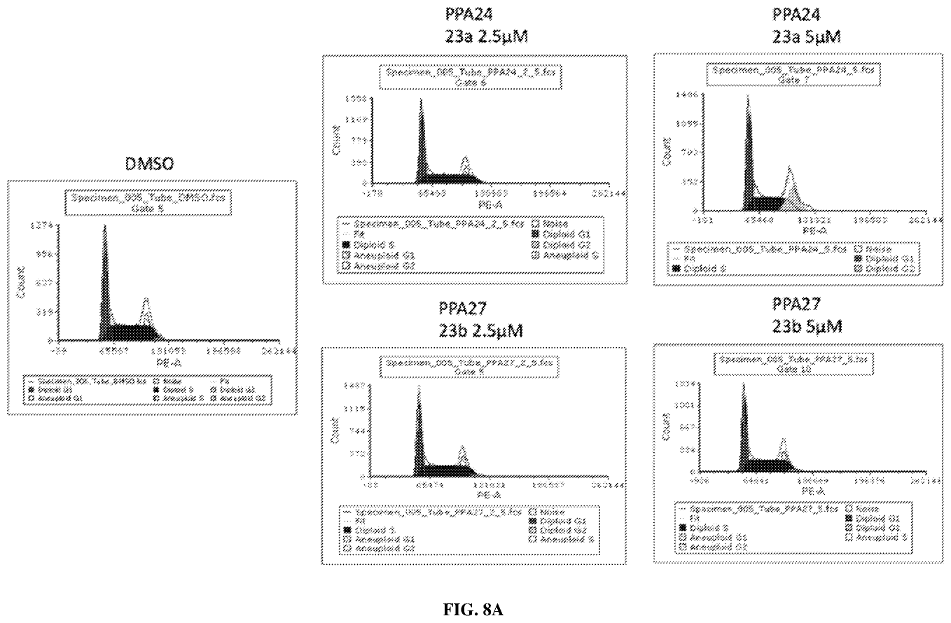

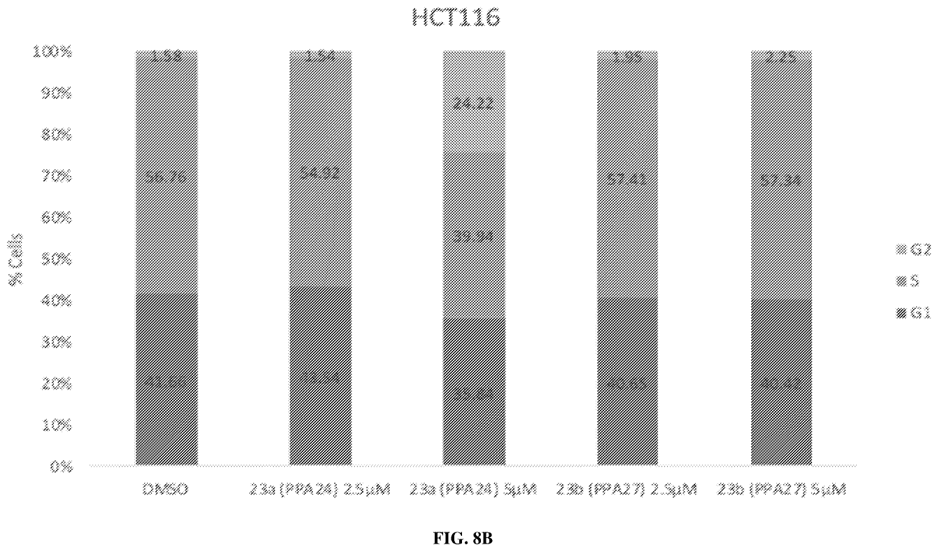

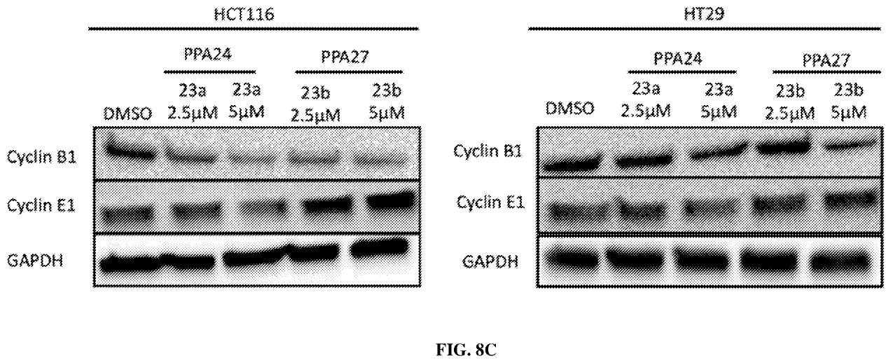

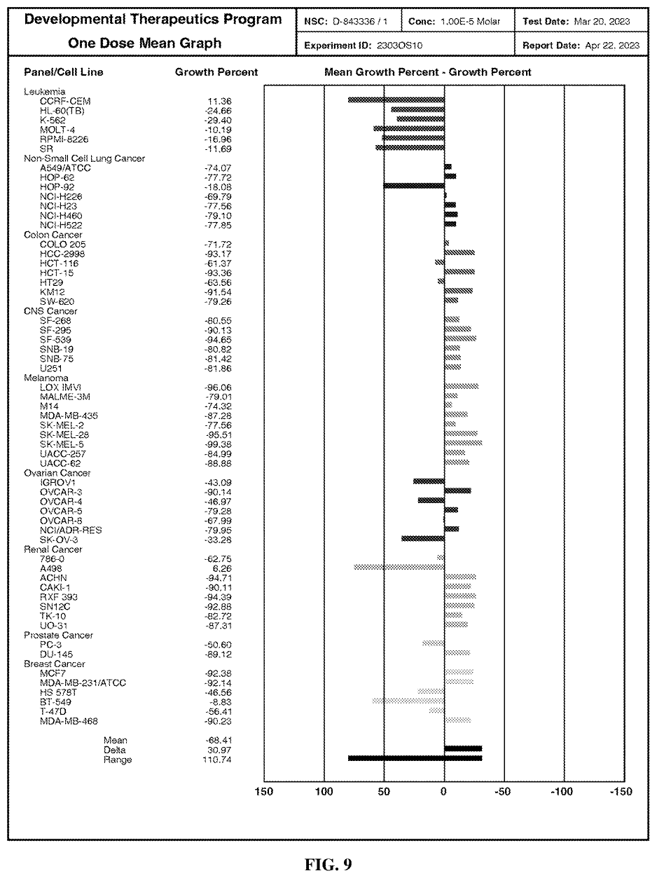

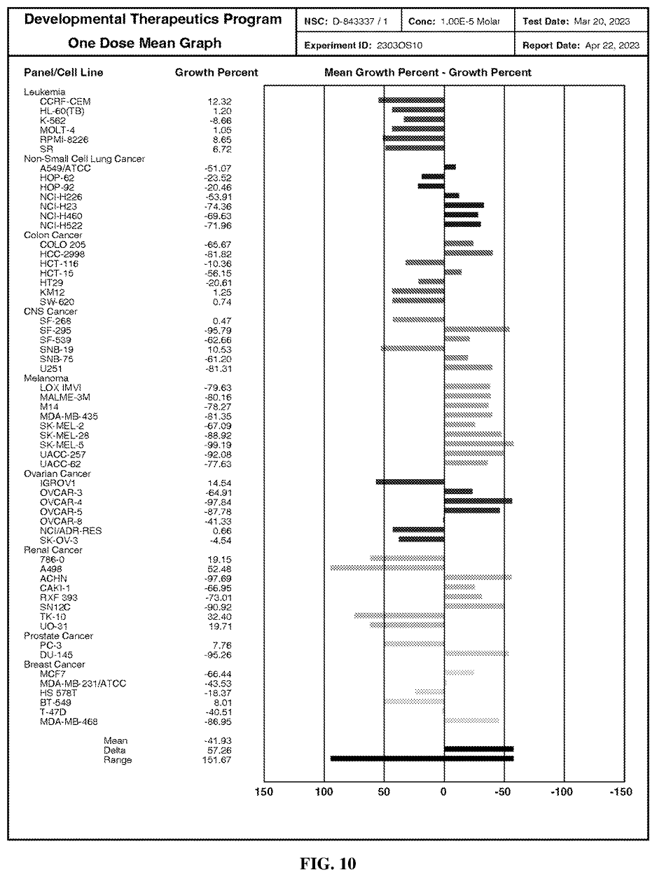

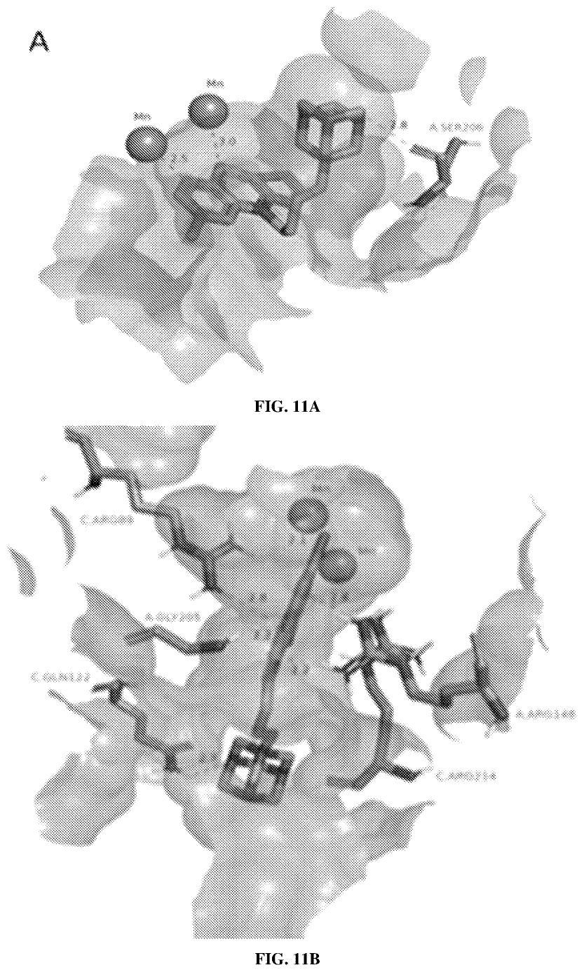

Compounds of the present disclosure can be made using standard chemical synthesis procedures and reagents known to one of ordinary skill in the art and as described herein. Representative synthetic schemes are shown in FIG. 2.

### Cellular and Enzymatic Assays

Compounds of the present disclosure were screened via MTS assay for 48 hrs at varying concentrations against different colon cancer cell lines (FIG. 3). The compounds were screened in four different colon cancer cell lines (HCT116, HT29, SW480, and DLD1) and normalized to DMSO. The cytotoxicity of PPA24, PPA27, iHAP1, and NSC49L in CRC and FOLFOX-resistant CRC cells (FOLFOX HCT116, FOLFOX HT29, HT29, HCT116, SW480, and DLD1) was also determined, and IC₅₀ values calculated from MTS assays using concentrations from 0.5-50 μM.

PP2A activity of PPA24, PPA27, iHPA1, and NSC49L was measured via Malachite green assay using recombinant PP2Ac protein and a threonine phosphopeptide as the substrate. The compounds were treated at concentrations of 250 and 500 nM. The change in color was measured by spectrophotometer at 630 nm. Analysis was completed using GraphPad Prism 7.0 software.

Apoptosis was measured in CRC cells after treatment with PPA24, PPA27, iHPA1, and NSC49L. All the cells were treated with PPA-24, PPA-27, iHAP1, and NSC49L at concentrations of 2.5, 5, and 10 μM for 24 hrs. Apoptosis was measured through either Caspase 3/7 and 7-AAD staining or Annexin V and 7-AAD staining. Apoptosis measurement was obtained using a Muse Cell Analyzer.

The cell cycles of CRC cells treated with PPA24 and PPA27 were analyzed. All cells were serum starved for 24 hrs. before treatment. Cell cycle arrest in HCT116 treated with PPA-24 and PPA-27 at 2.5 and 5 μM for 30 hrs was determined through flow cytometry. Analysis was completed using FCS Express 7.

### Molecular Modeling

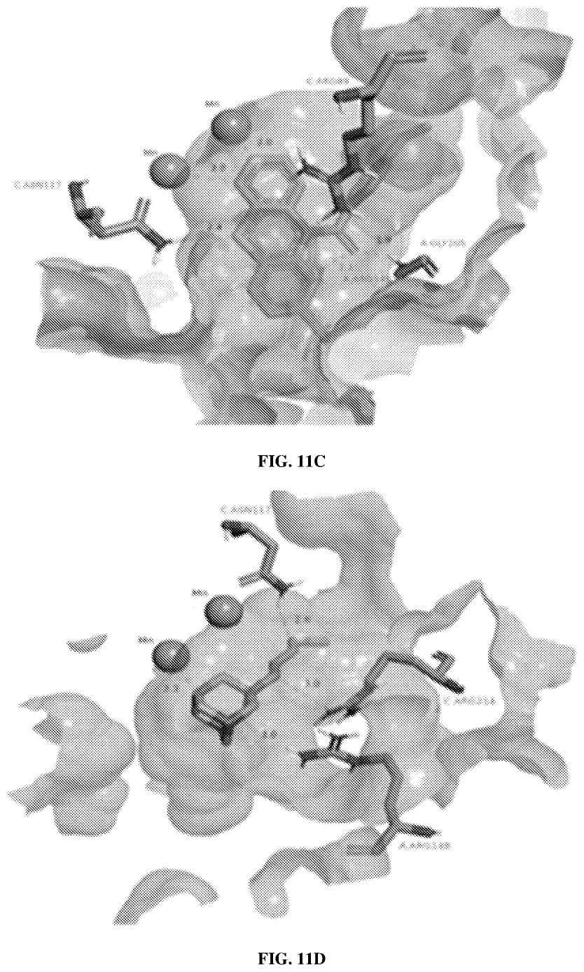

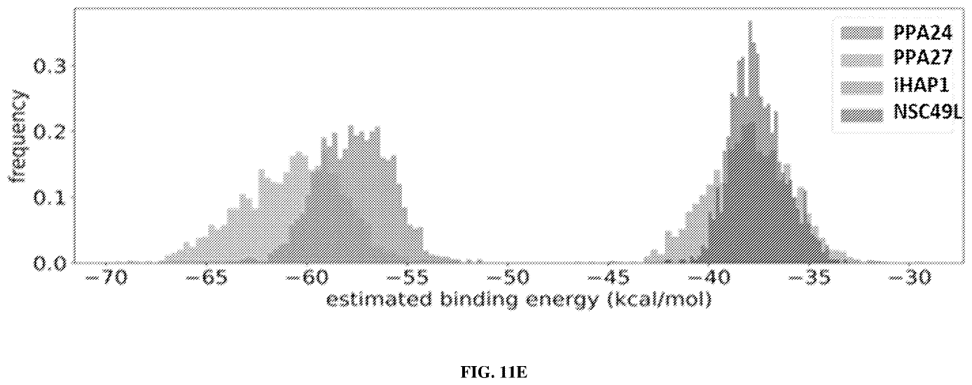

For further evaluation of the PP2A activity produced by PPA24 and PPA27 in comparison to iHAP1 and NSC49L, the binding of the four compounds to PP2A was compared through molecular modeling (PDB ID 4LAC) using MedusaDock. Compounds PPA24 and PPA27 had significantly lower binding energies than iHAP1 and NSC49L (FIGS. 11A-11E).

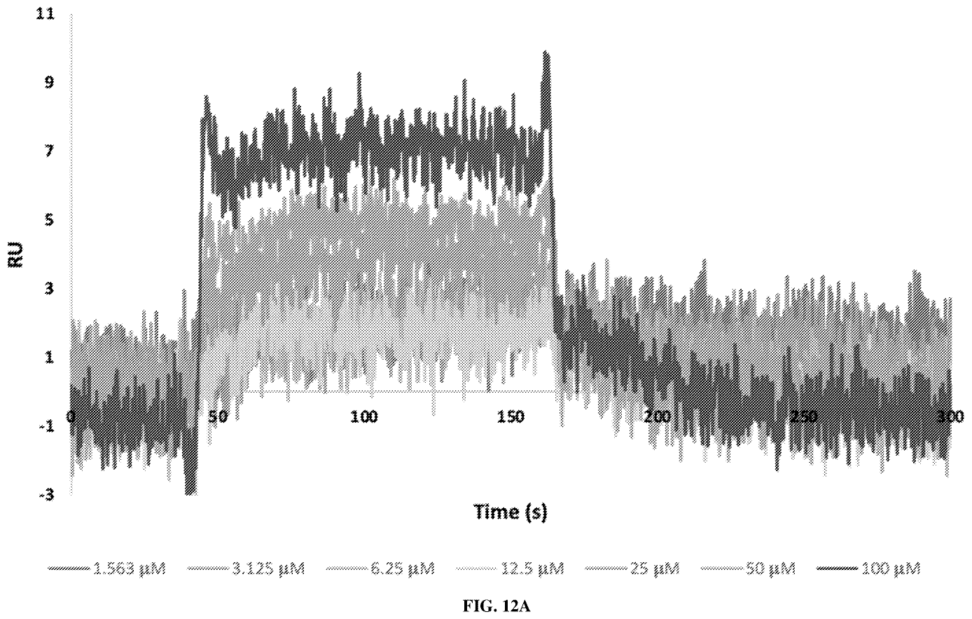

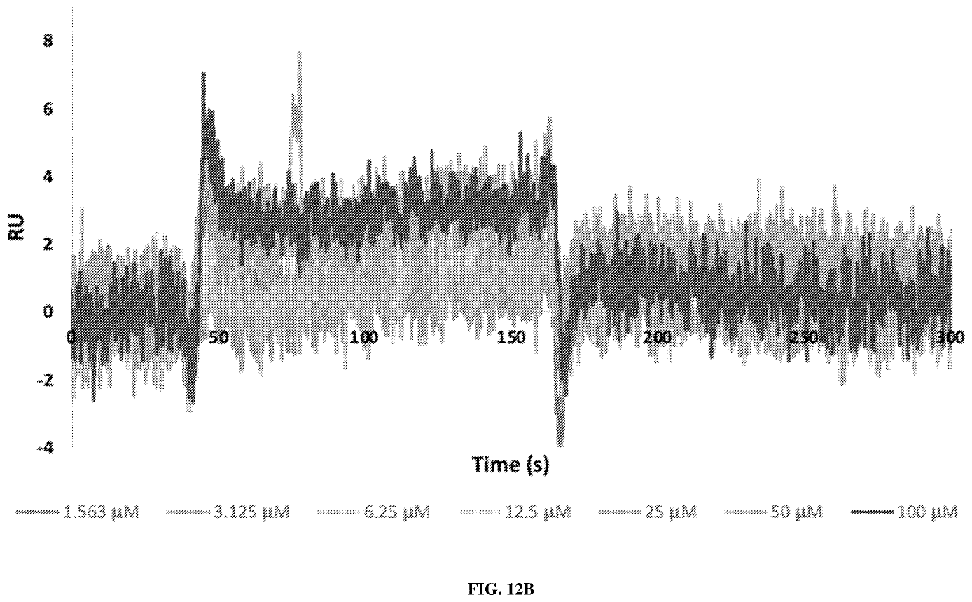

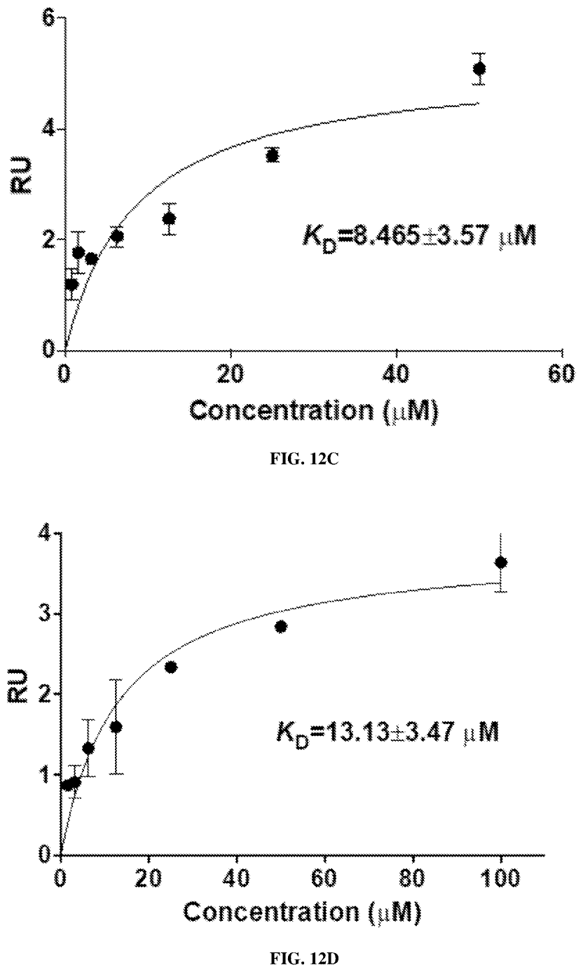

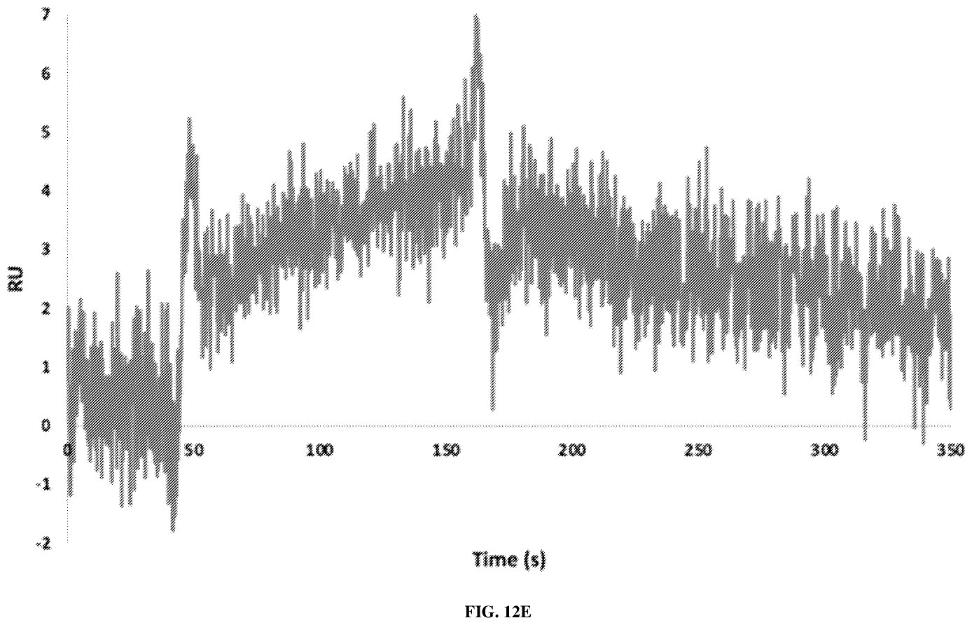

PPA24 showed the strongest binding to PP2ACα (Kd=8.465±3.57 μM). PPA27 (Kd=13.13±3.47 M) showed a relatively weaker binding. iHAP1 did not show a clear signal even at high concentrations and NSC49L only had a binding signal at 100 μM, consequently not allowing for the calculation of K_{D} for NSC49L. Thus, the binding affinity can be in the order of PPA24>PPA27>NSC49L>iHAP1. This data is consistent with the molecular docking results (FIGS. 12A-12E).

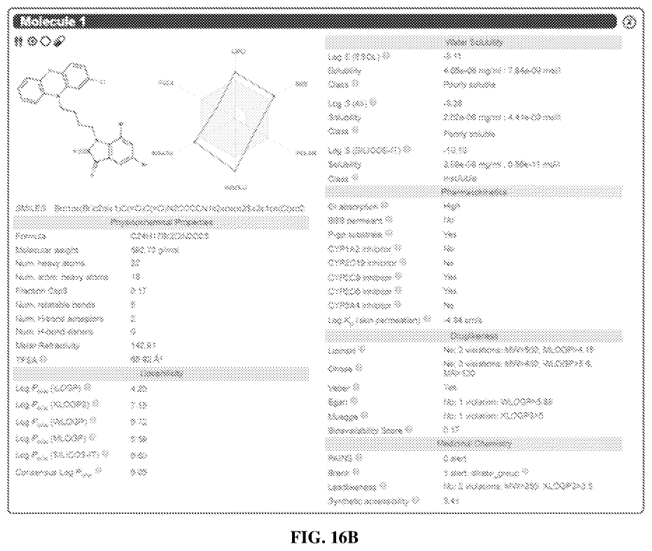

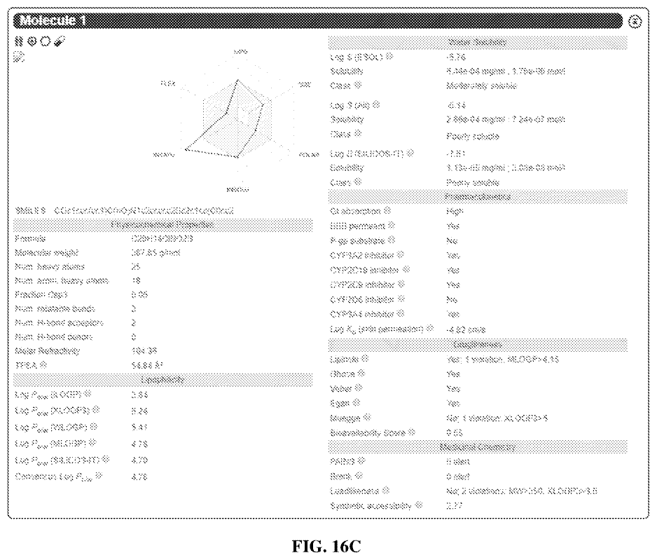

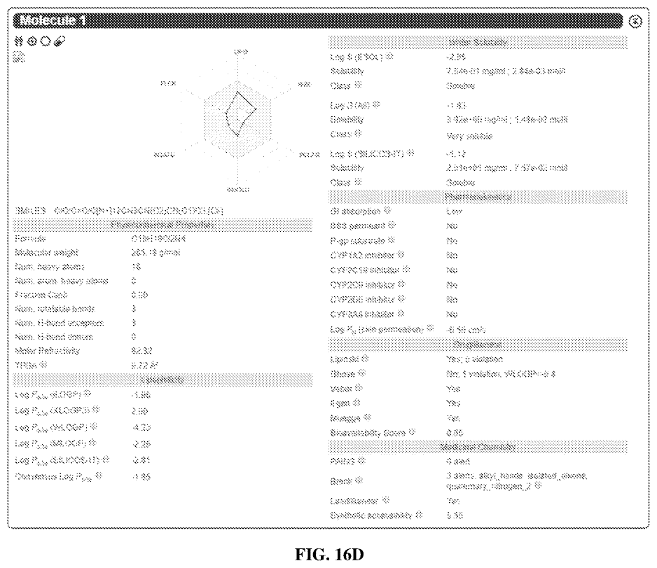

The pharmacological properties of PPA24, PPA27, iHAP1, and NSC49L were evaluated using SwissADME (FIGS. 16A-16D). SwissADME indicates the favorable ADME parameters, pharmacokinetic properties, and druglike nature of PPA24. PPA24 is a salt that has been found to be readily water soluble, as opposed to what is predicted by SwissADME.

### PPA and Reactive Oxygen Species

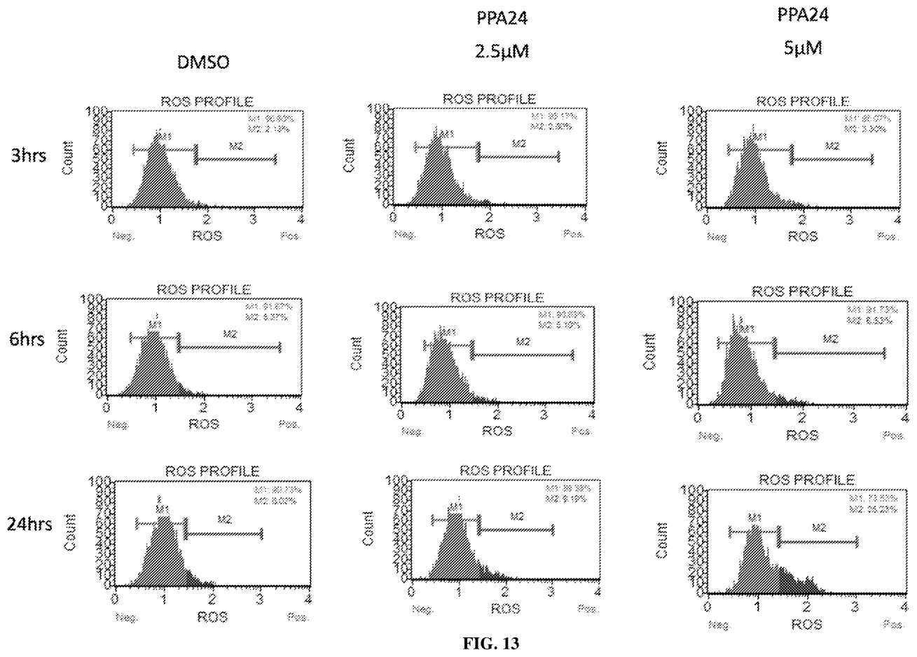

Though the exact role oxidative stress and PP2A play together is not known, in some cases reactive oxygen species (ROS) increase, decrease, or have no effect on PP2A activity. There have been links between PP2A activation leading to an increase in oxidative stress induced apoptosis. Due to this link, the ability of PPA24 to induce oxidative stress in HCT116 cells was investigated. It was found that after treatment with PPA24 for 24 hrs at 5 μM an increase in ROS positive cells occurred. This coincides with the previous apoptosis data indicating significant cell death occurring at these same conditions. (FIG. 13)

### Selectivity for Cancerous Cells

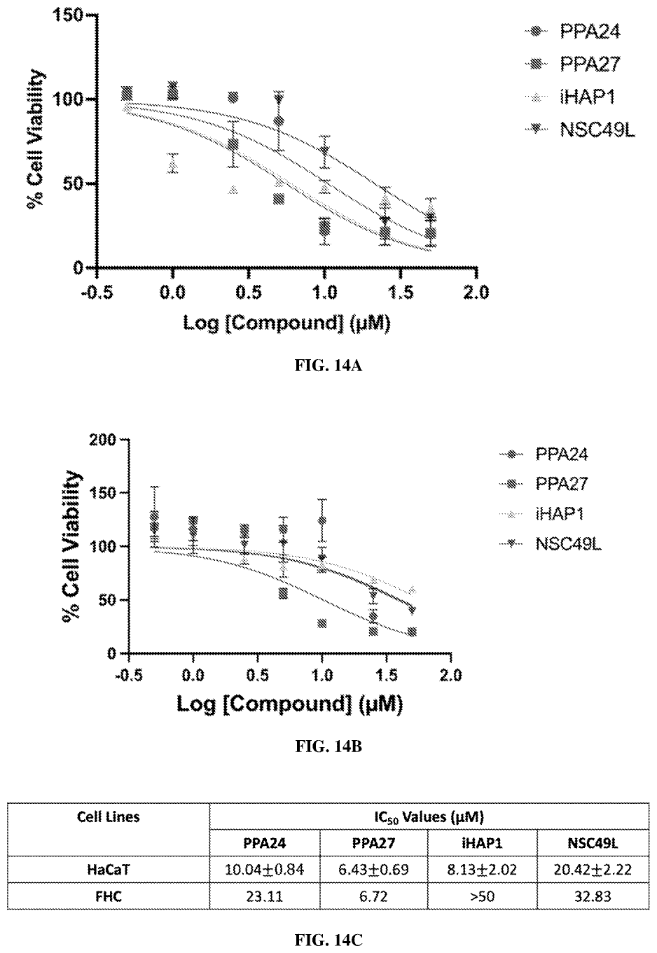

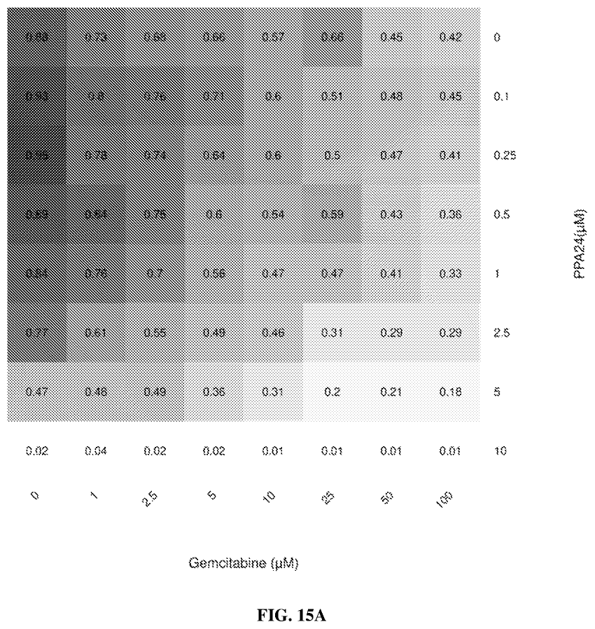

The compounds PPA24, PPA27, iHAP1, and NSC49L were screened in noncancerous cell lines to observe the selectivity these compounds have towards cancerous cells by assessing the differences in IC₅₀ concentrations. In the HaCaT cells, PPA24 was found to have an IC₅₀ concentration of 10.04±0.84 μM which is approximately 2-fold higher than the IC₅₀ concentration range seen in CRC cells. PPA27 and iHAP1 do not appear to have a significant difference regarding cell viability in these cells versus CRC cells; however, NSC49L does appear to have some selectivity towards cancerous cells based on these measures. The preliminary data presented in the FHC cells, indicated more tolerability towards all of the compounds, with the exception of PPA27, as compared to CRC and HaCaT cells (FIGS. 14A-14C).

### Synergistic Combination of PPA24 and Gemcitabine

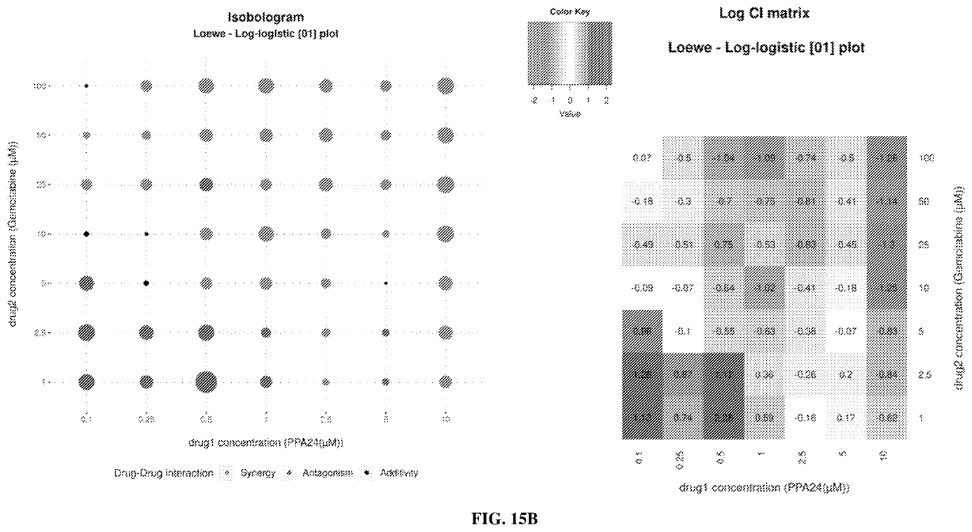

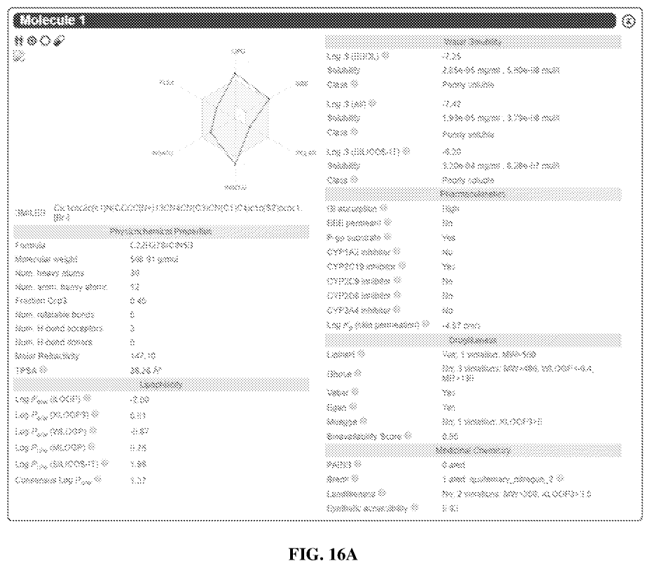

The combination of PPA24 and Gemcitabine, a clinically used cancer drug, in the CRC cells, worked synergistically to decrease the cell viability at lower concentrations than possible when used alone. In contrast, no synergy was observed in combination with oxaliplatin and 5-FU (FIGS. 15A-15B).

### INCORPORATION BY REFERENCE

The present application refers to various issued patent, published patent applications, scientific journal articles, and other publications, all of which are incorporated herein by reference. The details of one or more embodiments of the present disclosure are set forth herein. Other features, objects, and advantages of the present disclosure will be apparent from the Detailed Description, the Figures, the Examples, and the Claims.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I): 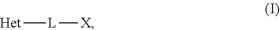
or a pharmaceutically acceptable salt thereof, wherein:
Het represents a heterocyclyl group;
L represents a linker; and
X represents a positively charged, bulky group.

2. The compound according to any preceding claim, or a pharmaceutically acceptable salt thereof, wherein X is of Formula (II): 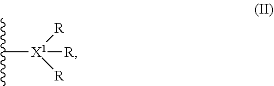
wherein:
X¹ is N or C; and
each instance of R is independently selected from H, C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl, wherein any two or more instances of R can together form a ring;
wherein when X¹ is C, then at least one instance of R is a C₁₋₁₂ heteroalkyl group having at least one quaternary nitrogen atom.

3. The compound according to any preceding claim, or a pharmaceutically acceptable salt thereof, wherein no instance of R is hydrogen.

4. The compound according to any preceding claim, or a pharmaceutically acceptable salt thereof, wherein X is of Formula (II-a): 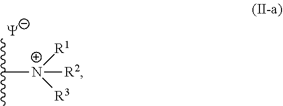
wherein:
R¹ is C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl;
R² is C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl;
R³ is C₁₋₁₂ alkyl, C₁₋₁₂ heteroalkyl, aryl, C₃₋₁₂ cycloalkyl, C₁₋₁₂ heterocyclyl, or C₁₋₁₂ heteroaryl; and
Ψ represents an anionic group;
wherein any two or more of R¹, R², and R³ may together form a ring.

5. The compound according to any preceding claim, or a pharmaceutically acceptable salt thereof, wherein Ψ is covalently bound to the compound.

6. The compound according to any preceding claim, or a pharmaceutically acceptable salt thereof, wherein Ψ is selected from COOH, SO₃H, and PO₃H₂.

7. The compound according to any preceding claim, or a pharmaceutically acceptable salt thereof, wherein Ψ is not covalently bond to the compound.

8. The compound according to any preceding claim, or a pharmaceutically acceptable salt thereof, wherein Ψ is selected from halide, carboxylate, sulfate, nitrate, and phosphate.

9. The compound according to any preceding claim, or a pharmaceutically acceptable salt thereof, wherein X is of Formula (II-b): 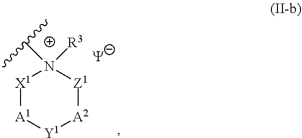
wherein:
X¹ is null, C₁₋₈ alkylene, or C₁₋₈ heteroalkylene;
Y¹ is null, C₁₋₈ alkylene, or C₁₋₈ heteroalkylene;
Z¹ is null, C₁₋₈ alkylene, or C₁₋₈ heteroalkylene;
A¹ is null, CHR⁴ or NR⁵;
A² is null, CHR⁶ or NR⁷;
R⁴ is H, C₁₋₈ alkyl, or C₁₋₈ heteroalkyl;
R⁵ is H, C₁₋₈ alkyl, or C₁₋₈ heteroalkyl;
R⁶ is H, C₁₋₈ alkyl, or C₁₋₈ heteroalkyl; and
R⁷ is H, C₁₋₈ alkyl, or C₁₋₈ heteroalkyl;
wherein any two or more of X¹, Y¹, Z¹, R³, R⁴, R⁵, R⁶, or R⁷ can together form a ring;
with the proviso that the compound includes at least one of X¹, Y¹, Z¹, and at least one of A or A².

10. The compound according to any preceding claim, or a pharmaceutically acceptable salt thereof, wherein A¹ is CHR⁴.

11. The compound according to any preceding claim, or a pharmaceutically acceptable salt thereof, wherein A¹ is NR⁵.

12. The compound according to any preceding claim, or a pharmaceutically acceptable salt thereof, wherein A² is CHR⁶.

13. The compound according to any preceding claim, or a pharmaceutically acceptable salt thereof, wherein A² is NR⁷.

14. The compound according to any preceding claim, or a pharmaceutically acceptable salt thereof, wherein Y¹ and A² are null.

15. The compound according to any preceding claim, or a pharmaceutically acceptable salt thereof, wherein X is of Formula (II-c): 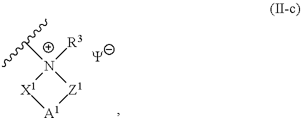
wherein:
X¹ is C₁₋₄ alkylene, preferably ethylene;
Z¹ is C₁₋₄ alkylene, preferably ethylene; and
A¹ is null, CHR⁴ or NR⁵;
wherein any two or more of X¹, Z¹, R³, R⁴, or R⁵ can together form a ring.

16. The compound according to any preceding claim, or a pharmaceutically acceptable salt thereof, wherein A¹ is CHR⁴.

17. The compound according to any preceding claim, or a pharmaceutically acceptable salt thereof, wherein R³ and R⁴ together form a ring.

18. The compound according to any preceding claim, or a pharmaceutically acceptable salt thereof, wherein R³ and R⁴ together form a C₁₋₄ alkylene group.

19. The compound according to any preceding claim, or a pharmaceutically acceptable salt thereof, wherein A¹ is NR⁵.

20. The compound according to any preceding claim, or a pharmaceutically acceptable salt thereof, wherein R³ and R⁵ together form a ring.

21. The compound according to any preceding claim, or a pharmaceutically acceptable salt thereof, wherein R³ and R⁵ together form a C₁₋₄ alkylene group.

22. The compound according to any preceding claim, or a pharmaceutically acceptable salt thereof, wherein X is of Formula (II-d): 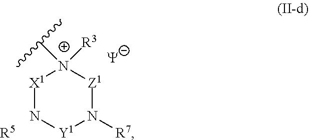
wherein:
X¹ is C₁₋₈ alkylene;
Y¹ is C₁₋₈ alkylene;
Z¹ is C₁₋₈ alkylene;
R⁵ is H, C₁₋₈ alkyl, or C₁₋₈ heteroalkyl; and
R⁷ is H, C₁₋₈ alkyl, or C₁₋₈ heteroalkyl,
wherein any two or more of X¹, Y¹, Z¹, R³, R⁵, and R⁷ can together form a ring.

23. The compound of any preceding claim, wherein R³ and R⁵ together form a ring.

24. The compound of any preceding claim, wherein R⁵ and R⁷ together form a ring.

25. The compound of any preceding claim, wherein R³, R⁵, and R⁷ together form a ring system.

26. The compound according to any preceding claim, or a pharmaceutically acceptable salt thereof, wherein:
X¹ is C₁₋₄ alkylene, preferably ethylene;
Y¹ is C₁₋₄ alkylene, preferably ethylene; and
Z¹ is C₁₋₄ alkylene, preferably ethylene.

27. The compound according to any preceding claim, or a pharmaceutically acceptable salt thereof, wherein X is of Formula (II-e): 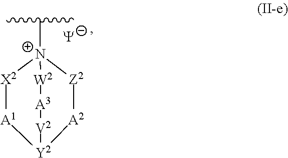
wherein:
X² is null, C₁₋₈ alkylene, or C₁₋₈ heteroalkylene;
A¹ is null, CHR⁴ or NR⁵;
wherein A¹ and X² cannot both be null;
Y² is N, C₁₋₈ alkylene, or C₁₋₈ heteroalkylene;
Z² is null, C₁₋₈ alkylene, or C₁₋₈ heteroalkylene;
A² is null, CHR⁶ or NR⁷;
wherein A² and Z² cannot both be null;
W² is null, C₁₋₈ alkylene, or C₁₋₈ heteroalkylene;
V² is null, C₁₋₈ alkylene, or C₁₋₈ heteroalkylene;
A³ is null, CHR⁸ or NR⁹;
wherein A², W², and V² cannot all be null,
R⁴ is H, C₁₋₈ alkyl, or C₁₋₈ heteroalkyl;
R⁵ is H, C₁₋₈ alkyl, or C₁₋₈ heteroalkyl;
R⁶ is H, C₁₋₈ alkyl, or C₁₋₈ heteroalkyl;
R⁷ is H, C₁₋₈ alkyl, or C₁₋₈ heteroalkyl;
R⁸ is H, C₁₋₈ alkyl, or C₁₋₈ heteroalkyl; and
R⁹ is H, C₁₋₈ alkyl, or C₁₋₈ heteroalkyl;
wherein any two or more of R⁴, R⁵, R⁶, R⁷, R⁸, and R⁹ can together form a ring.

28. The compound according to any preceding claim, or a pharmaceutically acceptable salt thereof, wherein X is of Formula (II-f): 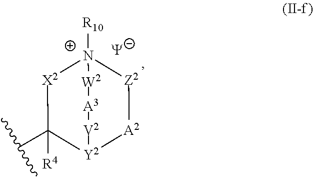
wherein:
X² is null, C₁₋₈ alkylene, or C₁₋₈ heteroalkylene;
Y² is N, C₁₋₈ alkylene, or C₁₋₈ heteroalkylene;
Z² is null, C₁₋₈ alkylene, or C₁₋₈ heteroalkylene;
A² is null, CHR⁶ or NR⁷;
wherein A² and Z² cannot both be null;
W² is null, C₁₋₈ alkylene, or C₁₋₈ heteroalkylene;
V² is null, C₁₋₈ alkylene, or C₁₋₈ heteroalkylene;
A³ is null, CHR⁸ or NR⁹;
wherein A², W², and V² cannot all be null;
R⁴ is H, C₁₋₈ alkyl, or C₁₋₈ heteroalkyl;
R⁶ is H, C₁₋₈ alkyl, or C₁₋₈ heteroalkyl;
R⁷ is H, C₁₋₈ alkyl, or C₁₋₈ heteroalkyl;
R⁸ is H, C₁₋₈ alkyl, or C₁₋₈ heteroalkyl;
R⁹ is H, C₁₋₈ alkyl, or C₁₋₈ heteroalkyl; and
R¹⁰ is C₁₋₈ alkyl or C₁₋₈ heteroalkyl;
wherein any two or more of R⁴, R⁶, R⁷, R⁸, and R⁹ can together form a ring.

29. The compound according to any preceding claim, or a pharmaceutically acceptable salt thereof, wherein X is of Formula (II-g): 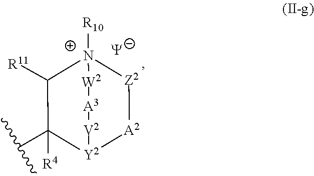
wherein:
Y² is N, C₁₋₈ alkylene, or C₁₋₈ heteroalkylene;
Z² is null, C₁₋₈ alkylene, or C₁₋₈ heteroalkylene;
A² is null, CHR⁶ or NR⁷;
wherein A² and Z² cannot both be null;
W² is null, C₁₋₈ alkylene, or C₁₋₈ heteroalkylene;
V² is null, C₁₋₈ alkylene, or C₁₋₈ heteroalkylene;
A³ is null, CHR⁸ or NR⁹;
wherein A², W², and V² cannot all be null;
R⁴ is H, C₁₋₈ alkyl, or C₁₋₈ heteroalkyl;
R⁶ is H, C₁₋₈ alkyl, or C₁₋₈ heteroalkyl;
R⁷ is H, C₁₋₈ alkyl, or C₁₋₈ heteroalkyl;
R⁸ is H, C₁₋₈ alkyl, or C₁₋₈ heteroalkyl;
R⁹ is H, C₁₋₈ alkyl, or C₁₋₈ heteroalkyl;
R¹⁰ is C₁₋₈ alkyl or C₁₋₈ heteroalkyl; and
R¹¹ is H, C₁₋₈ alkyl, or C₁₋₈ heteroalkyl;
wherein any two or more of R⁴, R⁶, R⁷, R⁸, R⁹, and R¹¹ can together form a ring.

30. The compound according to any preceding claim, or a pharmaceutically acceptable salt thereof, wherein X² is null.

31. The compound according to any preceding claim, or a pharmaceutically acceptable salt thereof, wherein A³ and V² are null.

32. The compound according to any preceding claim, or a pharmaceutically acceptable salt thereof, wherein A¹ is CHR⁴.

33. The compound according to any preceding claim, or a pharmaceutically acceptable salt thereof, wherein A¹ is NR⁵.

34. The compound according to any preceding claim, or a pharmaceutically acceptable salt thereof, wherein A² is CHR⁶.

35. The compound according to any preceding claim, or a pharmaceutically acceptable salt thereof, wherein A² is NR⁷.

36. The compound according to any preceding claim, or a pharmaceutically acceptable salt thereof, wherein A³ is CHR⁸.

37. The compound according to any preceding claim, or a pharmaceutically acceptable salt thereof, wherein A³ is NR⁹.

38. The compound according to any preceding claim, or a pharmaceutically acceptable salt thereof, wherein R⁴ and R⁶ together form a ring.

39. The compound according to any preceding claim, or a pharmaceutically acceptable salt thereof, wherein R⁵ and R⁶ together form a ring.

40. The compound according to any preceding claim, or a pharmaceutically acceptable salt thereof, wherein R⁴ and R⁷ together form a ring.

41. The compound according to any preceding claim, or a pharmaceutically acceptable salt thereof, wherein R⁵ and R⁷ together form a ring.

42. The compound according to any preceding claim, or a pharmaceutically acceptable salt thereof, wherein R⁴ and R⁸ together form a ring.

43. The compound according to any preceding claim, or a pharmaceutically acceptable salt thereof, wherein R⁵ and R⁸ together form a ring.

44. The compound according to any preceding claim, or a pharmaceutically acceptable salt thereof, wherein R⁴ and R⁹ together form a ring.

45. The compound according to any preceding claim, or a pharmaceutically acceptable salt thereof, wherein R⁸ and R⁹ together form a ring.

46. The compound according to any preceding claim, or a pharmaceutically acceptable salt thereof, wherein R⁶ and R⁸ together form a ring.

47. The compound according to any preceding claim, or a pharmaceutically acceptable salt thereof, wherein R⁷ and R⁸ together form a ring.

48. The compound according to any preceding claim, or a pharmaceutically acceptable salt thereof, wherein R⁶ and R⁹ together form a ring.

49. The compound according to any preceding claim, or a pharmaceutically acceptable salt thereof, wherein R⁷ and R⁹ together form a ring system.

50. The compound according to any preceding claim, or a pharmaceutically acceptable salt thereof, wherein R⁴, R⁶, and R⁸ together form a ring system.

51. The compound according to any preceding claim, or a pharmaceutically acceptable salt thereof, wherein R⁵, R⁷, and R⁹ together form a ring system.

52. The compound according to any preceding claim, or a pharmaceutically acceptable salt thereof, wherein R⁴ and R⁶ together form a C₁₋₄ alkylene group.

53. The compound according to any preceding claim, or a pharmaceutically acceptable salt thereof, wherein R⁵ and R⁶ together form a C₁₋₄ alkylene group.

54. The compound according to any preceding claim, or a pharmaceutically acceptable salt thereof, wherein R⁴ and R⁷ together form a C₁₋₄ alkylene group.

55. The compound according to any preceding claim, or a pharmaceutically acceptable salt thereof, wherein R⁵ and R⁷ together form a C₁₋₄ alkylene group.

56. The compound according to any preceding claim, or a pharmaceutically acceptable salt thereof, wherein R⁴ and R⁸ together form a C₁₋₄ alkylene group.

57. The compound according to any preceding claim, or a pharmaceutically acceptable salt thereof, wherein R⁵ and R⁸ together form a C₁₋₄ alkylene group.

58. The compound according to any preceding claim, or a pharmaceutically acceptable salt thereof, wherein R⁴ and R⁹ together form a C₁₋₄ alkylene group.

59. The compound according to any preceding claim, or a pharmaceutically acceptable salt thereof, wherein R⁵ and R⁹ together form a C₁₋₄ alkylene group.

60. The compound according to any preceding claim, or a pharmaceutically acceptable salt thereof, wherein R⁶ and R⁸ together form a C₁₋₄ alkylene group.

61. The compound according to any preceding claim, or a pharmaceutically acceptable salt thereof, wherein R⁷ and R⁸ together form a C₁₋₄ alkylene group.

62. The compound according to any preceding claim, or a pharmaceutically acceptable salt thereof, wherein R⁶ and R⁹ together form a C₁₋₄ alkylene group.

63. The compound according to any preceding claim, or a pharmaceutically acceptable salt thereof, wherein R⁷ and R⁹ together form a C₁₋₄ alkylene group.

64. The compound according to any preceding claim, or a pharmaceutically acceptable salt thereof, wherein R⁴, R⁶, and R⁸ together form a ring system having from 1-8 carbon atoms.

65. The compound according to any preceding claim, or a pharmaceutically acceptable salt thereof, wherein R⁵, R⁷, and R⁹ together form a ring system having from 1-8 carbon atoms.

66. The compound according to any preceding claim, or a pharmaceutically acceptable salt thereof, wherein X has the formula:

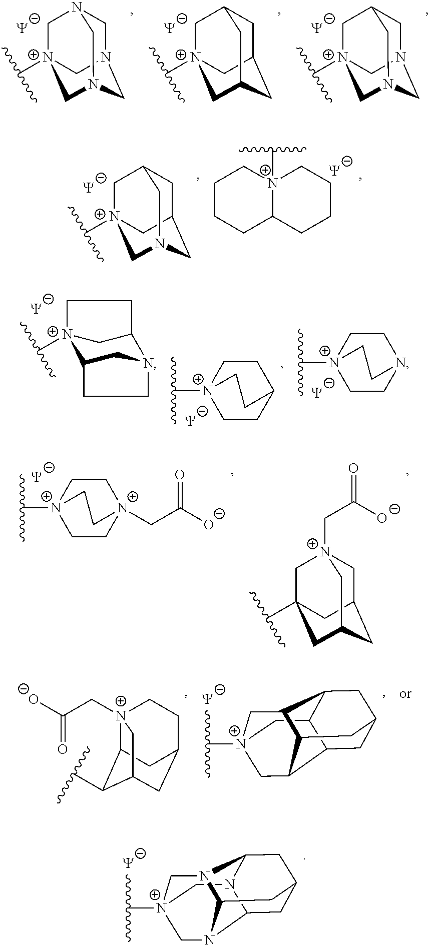

67. The compound according to any preceding claim, or a pharmaceutically acceptable salt thereof, wherein L comprises a C₁₋₁₂ alkylene, C₁₋₁₂ heteroalkylene, arylene, C₃₋₁₂ cycloalkylene, C₁₋₁₂ heterocyclylene, C₁₋₁₂ heteroarylene, polyethylene glycol, or a combination thereof.

68. The compound according to any preceding claim, or a pharmaceutically acceptable salt thereof, wherein L comprises a C₁₋₁₂ alkylene group.

69. The compound according to any preceding claim, or a pharmaceutically acceptable salt thereof, wherein L comprises a C₁₋₁₂ alkylene group comprising at least one double or triple bond.

70. The compound according to any preceding claim, or a pharmaceutically acceptable salt thereof, wherein L has the formula —(CH₂)ₙ—, wherein n is 1, 2, 3, 4, 5, 6, 7, 8, 9, or 10, preferably 2, 3, 4, 5, 6, 7, or 8, more preferably 2, 3, 4, 5, or 6, and especially preferably 3, 4, or 5.

71. The compound according to any preceding claim, or a pharmaceutically acceptable salt thereof, wherein L comprises a butylene group, optionally substituted one or more times by —OH, ═O, or —COOH.

72. The compound according to any preceding claim, or a pharmaceutically acceptable salt thereof, wherein L has the formula —CH₂CH₂CH₂CH₂—.

73. The compound according to any preceding claim, or a pharmaceutically acceptable salt thereof, wherein Het comprises one or more heterocyclyl groups selected from pyrrolyl, furanyl, thiophenyl, imidazolyl, pyrazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, triazinyl and tetrazinyl, azepinyl, oxepinyl, thiepinyl, indolyl, isoindolyl, isatinyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, purinyl, naphthyridinyl, pteridinyl, quinolinyl, isoquinolinyl, cinnolinyl, quinoxalinyl, phthalazinyl, quinazolinyl, phenanthridinyl, dibenzofuranyl, carbazolyl, acridinyl, phenothiazinyl, phenoxazinyl, alloxazinyl, dibenzazepinyl, dibenzoxazepinyl, thioxanthenyl, and phenazinyl.

74. The compound according to any preceding claim, or a pharmaceutically acceptable salt thereof, wherein Het is of Formula (III): 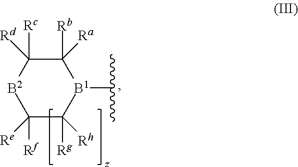
wherein:
z is 1 or 0;
B¹ is N, CR¹, C₂₋₃ alkylene, or C₁₋₃ heteroalkylene;
B² is null, O, S, NR^{j}, C(R^{k}R^{k}*), C(R^{k}R^{k}*)C(R^{l}R^{l}*), C(R^{k}R^{k}*)C(R^{l}R^{l}*)C(R^{m}R^{m}*) or C₁₋₃ heteroalkylene;
each of R^{a}, R^{b}, R^{c}, R^{d}, R^{e}, R^{f}, R^{g}, R^{h}, Rⁱ, R^{j}, R^{k}, R^{k}*, R^{l}, R^{l}*, R^{m}, and R^{m}* is independently selected from H, F, Cl, Br, I, OH, COOH, C(═O)O(C₁₋₆ alkyl), NH₂, N(C₁₋₆ alkyl)₂, NH(C₁₋₆ alkyl), C₁₋₆ alkyl, C₁₋₆ heteroalkyl, aryl, C₃₋₁₂ cycloalkyl, C₁₋₁₂ heterocyclyl, or C₁₋₁₂ heteroaryl, wherein said C₁₋₆ alkyl, C₁₋₁₂ heteroalkyl, aryl, C₃₋₁₂ cycloalkyl, C₁₋₁₂ heterocyclyl, and C₁₋₁₂ heteroaryl can be substituted one or more times by F, Cl, Br, I, OH, COOH, C(═O)O)C₁₋₄ alkyl), NH₂, N(C₁₋₄ alkyl)₂, NH(C₁₋₄ alkyl), C₁₋₄ alkyl, or C₁₋₄ heteroalkyl;
any two or more of R^{a}, R^{b}, R^{c}, R^{d}, R^{e}, R^{f}, R^{g}, R^{h}, Rⁱ, R^{j}, B¹, and B² can together form a ring;
any two geminal R groups can together form an oxo, a thiooxo, an imino, or an exo-olefin; and
any two R groups on adjacent carbon atoms can together form a double bond.

75. The compound according to any preceding claim, or a pharmaceutically acceptable salt thereof, wherein z is 0 and Het is of Formula (III-a): 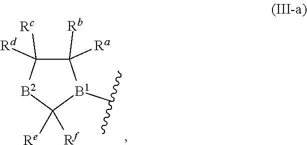
or z is 1 and Het is of Formula (III-b):

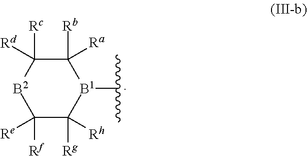

76. The compound according to any preceding claim, or a pharmaceutically acceptable salt thereof, wherein R^{a}, R^{b}, R^{c}, and R^{d} together form an aromatic or heteroaromatic ring of Formula (IV): 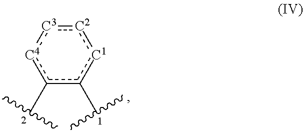
wherein
C¹ is null, O, S, N, CR^{Ar1}, or N(R^{Ar1}*)_{y};
C² is null, O, S, N, CR^{Ar2}, or N(R^{Ar2}*)_{y};
C³ is null, O, S, N, CR^{Ar3}, or N(R^{Ar3}*)_{y};
C⁴ is null, O, S, N, CR^{Ar4}, or N(R^{Ar4}*)_{y};
y is in each case selected from 0 or 1; [symbol]
represents a single or double bond;
wavy line 1 indicates the bond to B¹;
wavy line 2 indicates the bond to B²;
each of R^{Ar1}, R^{Ar2}, R^{Ar3}, and R^{Ar4} is independently selected from H, F, Cl, Br, I, COOH, OH, NH₂, C₁₋₄ alkyl, C₁₋₄ heteroalkyl, O(C₁₋₄ alkyl), O(C₁₋₄ heteroalkyl), NH(C₁₋₄ alkyl), NH(C₁₋₄ heteroalkyl), N(C₁₋₄ alkyl)(C₁₋₄ heteroalkyl), N(C₁₋₄ alkyl)₂, N(C₁₋₄ heteroalkyl)₂; and
each of R^{Ar1}*, R^{Ar2}*, R^{Ar3}*, and R^{Ar4}* is independently selected from H, C₁₋₄ alkyl, and C₁₋₄ heteroalkyl;
wherein any two or more of R^{Ar1}, R^{Ar2}, R^{Ar3}, and R^{Ar4} can together form a ring, and wherein any of R^{Ar1}, R^{Ar1}*, R^{Ar4}, or R^{Ar4}* can form a ring with any one or more of R^{e}, R^{f}, R^{g}, R^{h}, B¹, and B².

77. The compound according to any preceding claim, or a pharmaceutically acceptable salt thereof, wherein R^{a}, R^{b}, R^{c}, and R^{d} together form a fused benzo ring of Formula (IV-a):

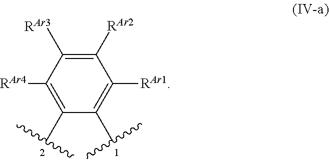

78. The compound according to any preceding claim, or a pharmaceutically acceptable salt thereof, wherein R^{e}, R^{f}, R⁹, and R^{h} together form an aromatic or heteroaromatic ring of Formula (V): 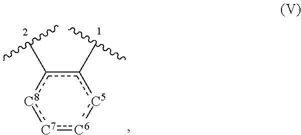
wherein
C⁵ is null, O, S, N, CR^{Ar5}, or N(RAr⁵*)_{y};
C⁶ is null, O, S, N, CR^{Ar6}, or N(RAr⁶*)_{y};
C⁷ is null, O, S, N, CR^{Ar7}, or N(RAr⁷*)_{y};
C⁸ is null, O, S, N, CR^{Ar8}, or N(RAr⁸*)_{y};
y is in each case selected from 0 or 1; [symbol]
represents a single or double bond;
wavy line 1 indicates the bond to B¹;
wavy line 2 indicates the bond to B²;
each of R^{Ar5}, R^{Ar6}, R^{Ar7}, and R^{Ar8} is independently selected from H, F, Cl, Br, I, COOH, OH, NH₂, C₁₋₄ alkyl, C₁₋₄ heteroalkyl, O(C₁₋₄ alkyl), O(C₁₋₄ heteroalkyl), NH(C₁₋₄ alkyl), NH(C₁₋₄ heteroalkyl), N(C₁₋₄ alkyl)(C₁₋₄ heteroalkyl), N(C₁₋₄ alkyl)₂, N(C₁₋₄ heteroalkyl)₂; and
each of R^{Ar5}*, R^{Ar6}*, R^{Ar7}*, and R^{Ar8}* is independently selected from H, C₁₋₄ alkyl, and C₁₋₄ heteroalkyl;
wherein any two or more of R^{Ar5}, R^{Ar6}, R^{Ar7}, and R^{Ar8} can together form a ring, and wherein any of R^{Ar5}, R^{Ar5}*, R^{Ar8}, or R^{Ar8}* can form a ring with any one or more of R^{a}, R^{b}, R^{c}, R^{d}, B¹, and B².

79. The compound according to any preceding claim, or a pharmaceutically acceptable salt thereof, wherein R^{e}, R^{f}, R⁹, and R^{h} together form a fused benzo ring of Formula (V-a)

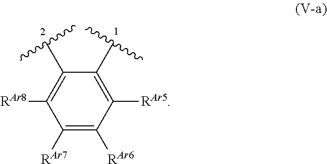

80. The compound according to any preceding claim, or a pharmaceutically acceptable salt thereof, wherein B² is null.

81. The compound according to any preceding claim, or a pharmaceutically acceptable salt thereof, wherein B² is NR^{j}.

82. The compound according to any preceding claim, or a pharmaceutically acceptable salt thereof, wherein B² is S.

83. The compound according to any preceding claim, or a pharmaceutically acceptable salt thereof, wherein B² is O.

84. The compound according to any preceding claim, or a pharmaceutically acceptable salt thereof, wherein Het is of Formula (III-a-1):

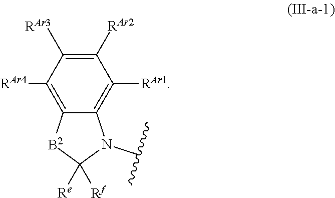

85. The compound according to any preceding claim, or a pharmaceutically acceptable salt thereof, wherein B² is NR^{j}; and R^{j} and R^{e} together form a double bond.

86. The compound according to any preceding claim, or a pharmaceutically acceptable salt thereof, wherein R^{e} and R^{f} together form an oxo.

87. The compound according to any preceding claim, or a pharmaceutically acceptable salt thereof, wherein B¹ is CRⁱ; Rⁱ and R^{e} together form a double bond; and B² is S, O, or NR^{j}.

88. The compound according to any preceding claim, or a pharmaceutically acceptable salt thereof, wherein R^{e} and R^{f} together form an oxo; B² is C(R^{k}R^{k}*); and R^{k} and R^{k}* together form an oxo.

89. The compound according to any preceding claim, or a pharmaceutically acceptable salt thereof, wherein Het is of Formula (III-b-1):

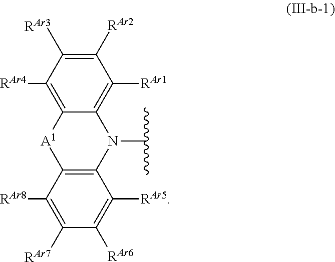

90. The compound according to any preceding claim, wherein the compound is of Formula (I-a): 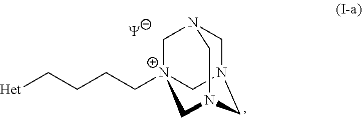
or a pharmaceutically acceptable salt thereof.

91. The compound according to any preceding claim, wherein the compound is of Formula (I-b): 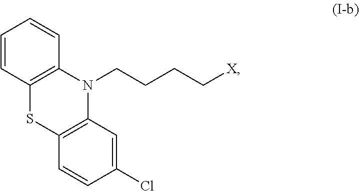
or a pharmaceutically acceptable salt thereof.

92. The compound according to any preceding claim, wherein the compound is of formula: 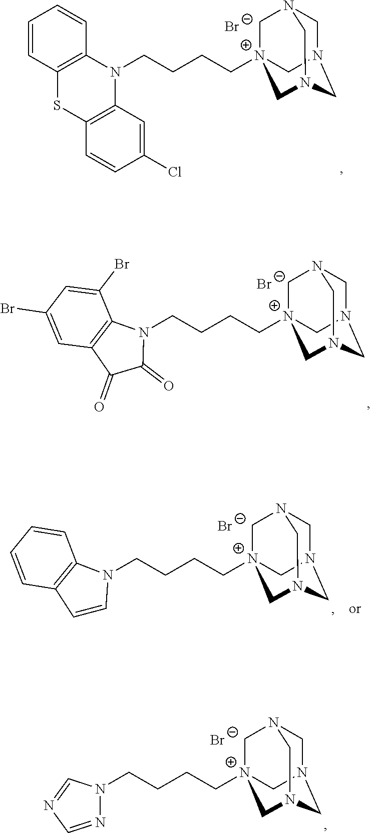
or a pharmaceutically acceptable salt thereof.

93. A pharmaceutical composition, comprising a compound according to any preceding claim, or a pharmaceutically acceptable salt thereof, and a pharmaceutically acceptable excipient.

94. The pharmaceutical composition according to any preceding claim, wherein the pharmaceutical composition comprises a second therapeutic agent.

95. The pharmaceutical composition according to any preceding claim, wherein the second therapeutic agent is a cancer chemotherapeutic.

96. A method of activating protein phosphatase 2A, comprising administering to a patient in need thereof a compound according to any preceding claim, or a pharmaceutically acceptable salt thereof.

97. A method of treating cancer, comprising administering to a patient in need thereof a compound according to any preceding claim, or a pharmaceutically acceptable salt thereof.

98. The method according to any preceding claim, wherein the cancer is acute lymphoblastic leukemia (ALL), acute myeloid leukemia (AML), cancer in adrenocortical carcinoma, adrenal cortex cancer, AIDS-related cancers, Kaposi sarcoma, AIDS-related lymphoma, primary CNS lymphoma, anal cancer, appendix cancer, carcinoid tumors, astrocytomas, atypical teratoid/rhabdoid tumor, basal cell carcinoma, skin cancer (nonmelanoma), bile duct cancer, extrahepatic bladder cancer, bladder cancer, bone cancer (includes Ewing sarcoma and osteosarcoma and malignant fibrous histiocytoma), brain tumors, breast cancer, bronchial tumors, Burkitt lymphoma (non-Hodgkin), carcinoid tumor, cardiac (heart) tumors, atypical teratoid/rhabdoid tumor, embryonal tumors, germ cell tumors, lymphoma, primary—cervical cancer, cholangiocarcinoma, chordoma, chronic lymphocytic leukemia (CLL), chronic myelogenous leukemia (CML), chronic myeloproliferative neoplasms, colorectal cancer, colorectal cancer, craniopharyngioma, cutaneous T-cell lymphoma, ductal carcinoma in situ (DCIS), embryonal tumors, central nervous system, endometrial cancer, ependymoma, esophageal, esthesioneuroblastoma, ewing sarcoma, extracranial germ cell tumor, extragonadal germ cell tumor, eye cancer, intraocular melanoma, retinoblastoma, fallopian tube cancer, fibrous histiocytoma of bone, malignant, and osteosarcoma, gallbladder cancer, gastric (stomach) cancer, gastric (stomach) cancer, gastrointestinal carcinoid tumor, gastrointestinal stromal tumors (GIST), gastrointestinal stromal tumors (GIST), germ cell tumors, central nervous system, extracranial, extragonadal, ovarian testicular, gestational trophoblastic disease, gliomas, hairy cell leukemia, head and neck cancer, heart tumors, hepatocellular (liver) cancer, histiocytosis, Langerhans Cell, Hodgkin's lymphoma, hypopharyngeal cancer, intraocular melanoma, islet cell tumors, pancreatic neuroendocrine tumors, Kaposi sarcoma, kidney—langerhans cell histiocytosis, laryngeal cancer, laryngeal cancer and papillomatosis, leukemia, lip and oral cavity cancer, liver cancer (primary), lung cancer, lung cancer, lymphoma—macroglobulinemia, Waldenström-Non-Hodgkin lymphoma, male breast cancer, malignant fibrous histiocytoma of bone and osteosarcoma, melanoma, intraocular (eye), Merkel cell carcinoma, mesothelioma, malignant, mesothelioma, metastatic squamous neck cancer with occult primary, midline tract carcinoma involving NUT gene, mouth cancer, multiple endocrine neoplasia syndromes, multiple myeloma/plasma cell neoplasms, mycosis fungoides, myelodysplastic syndromes, myelodysplastic/myeloproliferative neoplasms and chronic myeloproliferative neoplasms, myelogenous leukemia, chronic (CML), myeloid leukemia, acute (AML), nasal cavity and paranasal sinus cancer, nasopharyngeal cancer, nasopharyngeal cancer, neuroblastoma, non-hodgkin lymphoma, non-small cell lung cancer, oral cancer, lip and oral cavity cancer and oropharyngeal cancer, osteosarcoma and malignant fibrous histiocytoma of bone, ovarian cancer, pancreatic cancer and pancreatic neuroendocrine tumors (islet cell tumors), papillomatosis, paraganglioma, paraganglioma, paranasal sinus and nasal cavity cancer, parathyroid cancer, penile cancer, pharyngeal cancer, pheochromocytoma, pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, renal cell (kidney) cancer, retinoblastoma, rhabdomyosarcoma, salivary gland cancer, salivary gland tumors, Ewing sarcoma, Kaposi sarcoma, osteosarcoma, rhabdomyosarcoma, uterine sarcoma, vascular tumors, Sézary syndrome, skin cancer, small cell lung cancer, small intestine cancer, soft tissue sarcoma, squamous cell carcinoma, squamous neck cancer with occult primary, metastatic, stomach (gastric) cancer, stomach (gastric) cancer, T-cell lymphoma, cutaneous, testicular cancer, throat cancer, thymoma and thymic carcinoma, thyroid cancer, ureter and renal pelvis, transitional cell cancer, urethral cancer, uterine cancer, endometrial and uterine sarcoma, vaginal cancer, vaginal cancer, vascular tumors, vulvar cancer, Waldenström Macroglobulinemia, or Wilms Tumor.

99. The method according to any preceding claim, wherein the cancer is colorectal cancer.

100. The method according to any preceding claim, wherein the cancer is colorectal cancer that has been previously treated with 5-fluorouracil, folinic acid, oxaliplatin, or a combination thereof.

101. The method according to any preceding claim, wherein the cancer is colorectal cancer that has been previously treated with 5-fluorouracil, folinic acid, and oxaliplatin.

102. The method according to any preceding claim, wherein the cancer is colorectal cancer that is resistant to one or more of 5-fluorouracil, folinic acid, and oxaliplatin.

103. The method according to any preceding claim, further comprising administering to the patient at least one additional therapy.

104. The method according to any preceding claim, wherein the at least one additional therapy comprises surgery, radiation therapy, immunotherapy, chemotherapy, or a combination thereof.

105. The method according to any preceding claim, further comprising administering to the patient at least one additional therapeutic agent.

106. The method according to any preceding claim, wherein the at least one additional therapeutic agent comprises a cancer chemotherapeutic agent.

107. The method according to any preceding claim, wherein the at least one additional therapeutic agent comprises a nucleoside analogue, antifolate, antimetabolite, topoisomerase I inhibitor, anthracycline, podophyllotoxin, taxanes, vinca alkaloid, alkylating agent, platinum compound, proteasome inhibitor, nitrogen mustard, estrogen analogue, monoclonal antibody, tyrosine kinase inhibitor, mTOR inhibitor, retinoid, immunomodulatory agent, histone deacetylase inhibitor, other kinase inhibitor, metabolic inhibitors, microtubule inhibitors, or combination thereof.

108. The method according to any preceding claim, wherein the at least one additional therapeutic agent comprises abiraterone acetate, methotrexate, paclitaxel albumin-stabilized nanoparticle, brentuximab vedotin, ado-trastuzumab emtansine, doxorubicin hydrochloride, afatinib dimaleate, everolimus, netupitant, palonosetron hydrochloride, imiquimod, aldesleukin, alectinib, alemtuzumab, melphalan hydrochloride, melphalan, pemetrexed disodium, chlorambucil, aminolevulinic acid, anastrozole, aprepitant, pamidronate disodium, exemestane, nelarabine, arsenic trioxide, ofatumumab, asparaginase Erwinia chrysanthemi, atezolizumab, bevacizumab, axitinib, azacitidine, carmustine, belinostat, bendamustine hydrochloride, bevacizumab, bexarotene, tositumomab, bicalutamide, bleomycin, blinatumomab, blinatumomab, bortezomib, bosutinib, busulfan, cabazitaxel, cabozantinib, alemtuzumab, irinotecan hydrochloride, capecitabine, fluorouracil, carboplatin, carfilzomib, bicalutamide, lomustine, ceritinib, daunorubicin hydrochloride, cetuximab, chlorambucil, cyclophosphamide, clofarabine, cobimetinib, dactinomycin, cobimetinib, crizotinib, ifosfamide, ramucirumab, cytarabine, dabrafenib, dacarbazine, decitabine, daratumumab, dasatinib, daunorubicin hydrochloride, decitabine, efibrotide sodium, defibrotide sodium, degarelix, denileukin diftitox, denosumab, dexamethasone, dexrazoxane hydrochloride, dinutuximab, docetaxel, doxorubicin hydrochloride, dacarbazine, rasburicase, epirubicin hydrochloride, elotuzumab, oxaliplatin, eltrombopag olamine, aprepitant, elotuzumab, enzalutamide, epirubicin hydrochloride, cetuximab, eribulin mesylate, vismodegib, erlotinib hydrochloride, etoposide, raloxifene hydrochloride, melphalan hydrochloride, toremifene, panobinostat, fulvestrant, letrozole, filgrastim, fludarabine phosphate, flutamide, methotrexate, pralatrexate, obinutuzumab, gefitinib, gemcitabine hydrochloride, gemtuzumab ozogamicin, afatinib dimaleate, imatinib mesylate, glucarpidase, goserelin acetate, eribulin mesylate, trastuzumab, topotecan hydrochloride, palbociclib, ibritumomab tiuxetan, ibrutinib, ponatinib hydrochloride, idarubicin hydrochloride, idelalisib, imiquimod, axitinib, recombinant interferon alfa-2b, tositumomab, ipilimumab, gefitinib, romidepsin, ixabepilone, ixazomib citrate, ruxolitinib phosphate, cabazitaxel, ado-trastuzumab emtansine, palifermin, pembrolizumab, lanreotide acetate, lapatinib ditosylate, lenalidomide lenvatinib mesylate, leuprolide acetate, olaparib, vincristine sulfate, procarbazine hydrochloride, mechlorethamine hydrochloride, megestrol acetate, trametinib, mercaptopurine, temozolomide, mitoxantrone hydrochloride, plerixafor, busulfan, azacitidine, gemtuzumab ozogamicin, vinorelbine tartrate, necitumumab, nelarabine, sorafenib tosylate, nilotinib, ixazomib citrate, nivolumab, romiplostim, obinutuzumab, ofatumumab, olaparib, omacetaxine mepesuccinate, pegaspargase, ondansetron hydrochloride, osimertinib, panitumumab, panobinostat, peginterferon alfa-2b, pembrolizumab, pertuzumab, plerixafor, pomalidomide, ponatinib hydrochloride, necitumumab, pralatrexate, procarbazine hydrochloride, aldesleukin, denosumab, ramucirumab, rasburicase, regorafenib, lenalidomide, rituximab, rolapitant hydrochloride, romidepsin, ruxolitinib phosphate, siltuximab, dasatinib, sunitinib malate, thalidomide, dabrafenib, osimertinib, talimogene, atezolizumab, temsirolimus, thalidomide, dexrazoxane hydrochloride, trabectedin, trametinib, trastuzumab, lapatinib ditosylate, dinutuximab, vandetanib, rolapitant hydrochloride, bortezomib, venetoclax, crizotinib, enzalutamide, ipilimumab, trabectedin, ziv-aflibercept, idelalisib, ceritinib, or combination thereof.

109. The method according to any preceding claim, wherein the cancer chemotherapeutic agent is selected from 5-fluorouracil, capecitabine, irinotecan, oxaliplatin, trifluridine, tipiracil, and combinations thereof.
